SEQUENCE LISTING (4 sequences)

SEQ ID NO: 1 - DNA, 24 bp, Artificial Sequence
  Synthetic Primer
gcaaatccttgctgttccaaccca

SEQ ID NO: 2 - DNA, 24 bp, Artificial Sequence
  Synthetic Primer
ttggggatccgggatgaactctct

SEQ ID NO: 3 - DNA, 25 bp, Artificial Sequence
  Synthetic Primer
tggaaaagcccagctcctagaccct

SEQ ID NO: 4 - DNA, 25 bp, Artificial Sequence
  Synthetic Primer
ttcctcgtgggcttgaagtgtgaaa

(19) United States
(12) Patent Application Publication
(10) Pub. No.: US 2008/0026003 A1
(43) Pub. Date: Jan. 31, 2008

(54) Methods for inhibiting pain

(75) Inventors: Clifford J. Woolf, Newton, MA (US); Tarek A. Samad, Charlestown, MA (US); Ru-Rong Ji, Medford, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 11/649,475

(22) Filed: Jan. 4, 2007

Related U.S. Application Data
(62) Division of application No. 10/416,257, filed on Dec. 15, 2003, now abandoned.
Application No. 10/416,257 is a continuation-in-part of application No. 09/708,375, filed on Nov. 8, 2000, now abandoned.

(51) Int. Cl. A61K 39/00; A61P 25/04

(52) U.S. Cl. 424/198.1

(57) ABSTRACT

The invention provides methods for the treatment or prevention of pain. The invention also provides screening methods for determining whether a compound inhibits IL-1β activity in the central nervous system of a mammal.

### CROSS-REFERENCE TO RELATED APPLICATIONS

This application is a divisional of U.S. application Ser. No. 10/416,257, filed Dec. 15, 2003, which is the National Stage of International Application No. PCT/US01/47419, filed Nov. 8, 2001, which is a continuation-in-part of U.S. application Ser. No. 09/708,375, filed Nov. 8, 2000, all hereby incorporated by reference.

### STATEMENT AS TO FEDERALLY SPONSORED RESEARCH

This invention was funded by grants NS38253-01 and NS40698 from the National Institutes of Health. The government has certain rights in the invention.

### BACKGROUND OF THE INVENTION

In general, the invention features methods for treating or preventing pain by inhibiting IL-1β activity in the central nervous system of a mammal.

Inflammation occurs in response to tissue damage, such as damage resulting from trauma, lack of blood supply, hemorrhage, foreign bodies, chemicals, irritants, allergens, electricity, heat, cold, microorganisms, surgical operations, or ionizing radiation. Inflammation is associated with pain, including hypersensitivity at the site of injury (primary hyperalgesia), hypersensitivity in neighboring non-injured tissue (secondary hyperalgesia), and diffuse pain. Primary hyperalgesia is produced by a reduction in the threshold of nociceptor terminals, called peripheral sensitization. Prostanoids produced by cyclooxygenase (Cox) at the site of inflammation contribute to the development of peripheral sensitization through PKA-mediated phosphorylation of sodium channels in nociceptor terminals. This phosphorylation increases the excitability and reduces the pain threshold of the nociceptor terminals. Secondary hyperalgesia is produced by an increase in excitability of neurons in the spinal cord, called central sensitization. Diffuse pain, which is less well understood, may include muscle and joint pain, such as the pain characteristic of flu-like symptoms.

Two rate-limiting steps in the production of prostanoids that contribute to primary and secondary hyperalgesia are the release of arachidonic acid (AA) from membrane phospholipids, which is regulated by phospholipase A₂ (PLA₂) enzymes, and the conversion of AA to the prostanoid precursor prostaglandin H₂ (PGH₂) which is catalyzed by Cox. Two isoforms of Cox have been identified: Cox-1, which is present in many cell types and, in general, is constitutively expressed; and Cox-2, which is induced at the site of inflammation and is constitutively expressed in the kidney and part of the central nervous system. In general, the anti-inflammatory and analgesic benefits of non-steroidal anti-inflammatory drugs (NSAIDs) are derived from inhibition of Cox-2, and their side effects are derived from inhibition of Cox-1.

Improved treatments are needed for treating or preventing pain with less severe side-effects. In particular, more potent treatments that cause fewer gastrointestinal complications are needed.

### SUMMARY OF THE INVENTION

The purpose of the present invention is to provide improved methods for treating, reducing, or preventing pain. We discovered that peripheral inflammation induces IL-1β in the central nervous system, resulting in increased levels of central Cox-2 and prostanoids that contribute to pain sensitivity. We also demonstrated that inhibition of central IL-1β activity is more effective in reducing pain than inhibition of peripheral IL-1β activity and is as effective in reducing pain as inhibition of central Cox-2 activity; thus, inhibition of central IL-1β activity is an improved method for treating, reducing, or preventing pain. Additionally, the methods of the present invention allow the selection of compounds that decrease IL-1β activity in the central nervous system of a mammal and therefore facilitate the identification of novel therapeutics useful for reducing, stabilizing, or preventing pain. We also demonstrated that inhibitors of MAP kinases decrease the induction of Cox-2 and prostanoids; thus, compounds that inhibit the phosphorylation or activity of a MAP kinase can be administered to the periphery and/or the central nervous system of a mammal to treat, reduce, or prevent pain.

Accordingly, in a first aspect, the invention provides a method of treating, reducing, or preventing pain which involves contacting the central nervous system of a mammal (for example, a human) with a compound that decreases IL-1β activity in a dose adequate to treat, reduce, or prevent pain. In one preferred embodiment, the compound is directly administered to the central nervous system of the mammal, preferably intrathecally, intramedullarly, intracerebrally, intracerebroventricularly, intracranially, intraspinally, epidurally, or intraparietally. In another preferred embodiment, the compound crosses the blood-brain barrier of the mammal. According to this embodiment, the compound that crosses the blood-brain barrier may be administered intravenously, parenterally, subcutaneously, intramuscularly, ophthalmically, intraventricularly, intraperitoneally, intranasally, orally, topically, or by any other route sufficient to provide a dose adequate to prevent, reduce, or treat pain. Preferably, the compound is administered together with a pharmaceutically acceptable carrier to the mammal. In preferred embodiments, a compound that directly or indirectly inhibits the phosphorylation or activity of p38 and/or a compound that directly or indirectly inhibits the phosphorylation or activity of ERK is administered.

Additionally, a compound that directly or indirectly inhibits the phosphorylation or activity of a MAP kinase such as p38 or ERK can be administered to the periphery or to both the periphery and the central nervous system of a mammal to treat, reduce, or prevent pain. One such method involves contacting the periphery of a mammal (for example, a human) with a first compound that decreases the activity or phosphorylation of a first MAP kinase in a dose adequate to treat, reduce, or prevent pain. In one embodiment, the method further involves administering the first compound or a second compound that decreases the enzymatic activity or phosphorylation level of the first MAP kinase or a second MAP kinase to the central nervous system of the mammal. Exemplary MAP kinases are p38 and ERK. In a preferred embodiment, a compound that inhibits the phosphorylation or activity of p38 and a compound that inhibits the phosphorylation or activity of ERK are administered. In one preferred embodiment, the compound is directly administered to the central nervous system of the mammal, preferably intrathecally, intramedullarly, intracerebrally, intracerebroventricularly, intracranially, intraspinally, epidurally, or intraparietally. In another preferred embodiment, the compound crosses the blood-brain barrier of the mammal. According to this embodiment, the compound that crosses the blood-brain barrier may be administered intravenously, parenterally, subcutaneously, intramuscularly, ophthalmically, intraventricularly, intraperitoneally, intranasally, orally, topically, or by any other route sufficient to provide a dose adequate to prevent, reduce, or treat pain. Preferably, the compound is administered together with a pharmaceutically acceptable carrier to the mammal. Preferably, the level of phosphorylation or enzymatic activity of the MAP kinase is at least 2, 3, 5, 10, 20, or 50-fold lower in the presence of the compound.

In addition, the present invention involves methods that may be used to determine whether a compound inhibits IL-1β activity in the central nervous system. Compounds identified using these methods may be useful in the treatment, reduction, or prevention of pain.

In one such aspect, the invention provides a screening method for determining whether a compound inhibits IL-1β activity in the central nervous system of a mammal that involves administering the compound to the periphery of the mammal, and measuring pain in the mammal or measuring IL-1β activity in the central nervous system of the mammal, in the presence and absence of the compound. The compound is determined to inhibit IL-1β activity if the compound effects a decrease in pain or IL-1β activity.

In another aspect, the invention provides a screening method for determining whether a compound selectively inhibits IL-1β activity in the central nervous system of a mammal. This method involves administering the compound to the periphery of the mammal and measuring IL-1β activity in both the central nervous system and the periphery of the mammal, in the presence and absence of the compound. The compound is determined to selectively inhibit IL-1β activity in the central nervous system if the compound effects a greater decrease in IL-1β activity in the central nervous system than in the periphery of the mammal.

In a related aspect, the invention provides yet another screening method for determining whether a compound selectively inhibits IL-1β activity in the central nervous system of a mammal. This method involves (a) administering the compound to the periphery of a first mammal, (b) measuring IL-1β activity in the periphery of the first mammal, in the presence and absence of the compound, (c) administering the compound to the central nervous system of the first mammal or a second mammal, and (d) measuring IL-1β activity in the central nervous system of that first mammal or second mammal, in the presence and absence of the compound. The compound is determined to selectively inhibit IL-1β activity in the central nervous system if the compound effects a greater decrease in IL-1β activity in the central nervous system than in the periphery.

In still another related aspect, the invention provides a screening method for determining whether a compound selectively inhibits IL-1β activity in the central nervous system of a mammal. This method includes (a) administering the compound to the periphery of a first mammal, (b) measuring pain in the first mammal, in the presence and absence of the compound, (c) administering the compound to the central nervous system of the first mammal or a second mammal, and (d) measuring the pain in that first mammal or second mammal, in the presence and absence of the compound. The compound is determined to selectively inhibit IL-1β activity in the central nervous system if the compound effects a greater decrease in pain when administered to the central nervous system than to the periphery.

In preferred embodiments of the various screening methods of the invention, the methods also include inducing inflammation in the periphery of the mammal, the first mammal, or the second mammal prior to measuring IL-1β activity or pain. Inflammation may be induced before, during, or after the administration of the compound to the periphery or the central nervous system. In other preferred embodiments, the methods also include inducing a nerve injury, lesion, or damage to cause neuropathic pain constituting a neuropathy, in the mammal, the first mammal, or the second mammal prior to measuring IL-1β activity or pain. Neuropathy may be induced before, during, or after the administration of the compound to the periphery or the central nervous system. Preferably, the compound is administered to the periphery of a mammal by intravenous, parenteral, subcutaneous, intramuscular, ophthalmic, intraventricular, intraperitoneal, oral, topically, or intranasal administration or to the central nervous system of a mammal by intrathecal, intramedullar, intracerebral, intracerebroventricular, intracranial, intraspinal, epidurally, or intraparietal administration. In other preferred embodiments, the compound is a member of a library of at least 5, 10, 20, 50, or 100 compounds, all of which are simultaneously administered to the mammal. In yet other preferred embodiments, the compound is administered together with a pharmaceutically acceptable carrier. Preferably, the mammal is a rodent, such as a mouse or rat; a monkey; a rabbit; or a guinea pig. Preferred compounds that inhibit central IL-1β activity include IL-1 receptor antagonists, such as recombinant IL-1ra, and caspase-1 inhibitors. Preferred caspase-1 inhibitors include, aldehydes, halomethylketones, diazomethylketones, phenylalkylketones, and acyloxymethylketones (Livingston, J. of Cellular Biochemistry 64:19-26, 1997; Calbiochem Technical Bulletin entitled Caspase Inhibitors and Substrates, San Diego, Calif.). Preferred aldehyde capase-1 inhibitors include acetyl-Tyr-Val-Ala-Asp-aldehyde, acetyl-Val-Ala-Asp-aldehyde, and acetyl-Ala-Ala-Val-Ala-Leu-Leu-Pro-Ala-Val-Leu-Leu-Ala-Leu-Leu-Ala-Pro-Tyr-Val-A la-Asp-aldehyde. Preferred halomethylketones include acetyl-Tyr-Val-Ala-Asp-chloromethylketone, Boc-Asp(O-methyl)-fluoromethylketone, Boc-Ala-Asp(O-benzyl)-chloromethylketone, Boc-Asp(O-benzyl)-chloromethylketone, benzoyloxycarbonyl-Tyr-Val-Ala-Asp(O-methyl)-fluoromethylketone, benzoyloxycarbonyl-Val-Ala-Asp-fluoromethylketone, and benzoyloxycarbonyl-Val-Ala-Asp(O-methyl)-fluoromethylketone. The halogen in any of these halomethylketones may be replaced with fluorine, chlorine, bromine, iodine, or astatine. Acetyl-Tyr-Val-Ala-Asp-diazomethylketone is a preferred diazomethylketone, and benzoyloxycarbonyl-Val-Ala-Asp-phenylalkylketones are preferred phenylalkylketones. Preferred examples of acyloxymethylketones include acetyl-Tyr-Val-Ala-Asp-[(2,6-dimethylbenzyoyl)oxy]methylketone, benzoyloxycarbonyl-Asp-CH₂-[(2,6-dichlorobenzoyl)oxy]methane, acetyl-Tyr-Val-Ala-Asp-(dichlorobenzoyl)oxy-methylketone, and benzoyloxycarbonyl-Val-Ala-Asp(O-ethyl)-(dichlorobenzoyl)oxy-methylketone. Derivatives of any of these compounds, including compounds in which the aspartic acid residue has been esterified using standard methods, may also be used in the methods of the invention.

In preferred embodiments of the various methods of the invention, the compounds directly or indirectly inhibits the phosphorylation or activity of a MAP kinase such as p38 or ERK or a transcription factor such as CREB. In other preferred embodiments, the compound indirectly or directly modulates the post-translational regulation (e.g., inhibits phosphorylation) of a protein downstream of IL-1β, p38, and/or ERK activation, such as a membrane receptor (e.g., the NMDA or AMPA receptor) or ion channel. Preferably, this inhibition of protein phosphorylation reduces membrane excitability, as measured using standard methods. In other preferred embodiments, administration of a compound that inhibits ERK activity or phosphorylation leads to a decreased level of phosphorylation of a transcription factor such as CREB or leads to a decreased level of gene transcription. In particular embodiments that result in decreased gene transcription, the compound effects a decrease in transcription of an immediate early gene such as c-fos or transcription of genes operably linked to a promoter containing a CRE-site (e.g., the prodynorphin or NK-1 promoter). In other embodiments, the mRNA or protein levels of prodynorphin and/or NK-1 are decreased by administration of the compound. In other embodiments, the compound causes a decrease in the transcription of an mRNA or in the half-life of an mRNA or protein, such as VR1.

As used herein, by “compound that decreases IL-1β activity” is meant a compound that decreases the level of IL-1β mRNA or protein, an activity of IL-1β, the half-life of IL-1β mRNA or protein, or the binding of IL-1β to a receptor or to another molecule, as measured using standard methods (see, for example, Ausubel et al., Current Protocols in Molecular Biology, Chapter 9, John Wiley & Sons, New York, 2000). In other preferred embodiments, a compound that decreases IL-1β activity reduces or stabilizes the level of Cox-2 mRNA or protein, the level of a prostanoid, the phosphorylation level of a signal transduction protein (e.g., a MAP kinase such as p38 or ERK), or the level or duration of pain. Cox-2 mRNA expression levels may be determined using standard RNase protection assays or in situ hybridization assays, such as those described herein, and the level of Cox-2 protein may be determined using standard Western or immunohistochemistry analysis with a Cox-2 antibody (see, for example, Ausubel et al., supra). Alternatively, the level of a prostanoid which is induced IL-1β, such as PGH₂ or PGE₂, may be measured using standard ELISA assays such as those described herein. The phosphorylation levels of signal transduction proteins downstream of IL-1 receptor activation, such as p38 MAP kinase, ERK MAP kinase, jun kinase (JNK), NFκ-B, or Iκ-B, may also be measured as described previously (O'Neill and Greene, J. Leukoc. Biol. 63:650-657, 1998; Auron, Cytokine Growth Factor Rev. 9:221-237, 1998). In various embodiments, the compound directly or indirectly inhibits the phosphorylation or activity of a MAP kinase such as p38 or ERK. In other embodiments, the compound does not directly or indirectly inhibit the phosphorylation or activity of a MAP kinase such as p38 or ERK. The level of IL-1β activity may be determined by measuring the level, duration, or delayed onset of pain as described below. Compounds that may be tested for their ability to decrease IL-1β activity include, but are not limited to, synthetic organic molecules, naturally occurring organic molecules, nucleic acid molecules, IL-1β antisense nucleic acids, biosynthetic proteins or peptides, naturally occurring peptides or proteins, IL-1β antibodies, or dominant negative IL-1β proteins. Preferably, the compound decreases IL-1β activity by at least 20, 40, 60, 80, or 90%. In another preferred embodiment, the level of IL-1β activity is at least 2, 3, 5, 10, 20, or 50-fold lower in the presence of the compound. Preferably, the decrease in IL-1β activity in the central nervous system is at least 2, 3, 5, 10, 20, or 50-fold greater than the decrease in IL-1β activity in the periphery.

By “pain” is meant a sensation of suffering due to some form of stimulation of nerve endings. Pain may be characterized by its location, quality (e.g., local, diffuse, constant, intermittent, burning, shooting, gnawing, sharp, dull, or throbbing), radiation (e.g., the distribution of the pain from the most severely affected location), frequency, or associated symptoms (e.g, mechanical threshold, or withdrawal latency). The level, duration, or delayed onset of pain in a mammal may be measured using any standard method. Preferred models of inflammatory pain include unilateral injection of formalin, carrageenan, or complete Freund's adjuvant (CFA) into the hindpaw of rodent, such as a rat or mouse (Honore et al., J. Neurosci. 19:7670-7678, 1999). For a model of chronic inflammatory pain, CFA may be used to induce arthritis in mice or rats (Honore et al., supra; Vieira et al., Eur. J. Pharmacol. 407:109-116, 2000). To measure the level of pain in these inflammatory pain models, withdrawal latency in response to a painful (heat) stimulus or mechanical hypersensitivity using calibrated von Frey filaments may be assayed as described previously (Decosterd et al., Pain 87:149-158, 2000). Alternatively, the amount of paw licking after intradermal or topical administration of an irritant, such as capsaicin, to the mouse or rat paw or the amount of wiping movements after local application of an irritant to the guinea pig conjunctiva may be measured (Vieira et al., supra). The ability of a compound to reduce, stablize, prevent, or delay the onset of pain may also be determined by measuring the effect of the compound on the amount of writhing by a rodent after intraperitoneal administration of acetic acid (Satumino et al., Biol. Pharm. Bull. 23:654-656, 2000). The length of avoidance by a CFA-treated rat of the location of a test chamber associated with mechanical stimulation of the inflamed paw may also be determined (LaBuda and Fuch, Exp. Neruol. 163:490-494, 2000). In one preferred embodiment, pain is measured at the site of injury (primary hyperalgesia) or in neighboring non-injured tissue (secondary hyperalgesia). In another preferred embodiment, diffuse pain is measured. In other preferred embodiments, hyperalgesia or allodynia is inhibited. It is also contemplated that pain originating in the central nervous system, such as pain after spinal cord injury, may be measured. In order to study peripheral neuropathic pain, for example, the ability of a compound to inhibit or prevent peripheral neuropathic pain can be measured in the Spared Nerve Injury model (Decosterd and Woolf, Pain 87:149-158, 2000) or the Chronic Constriction Injury model (Bennett and Xie, Pain 33:87-107, 1988).

By “periphery” or “peripheral nervous system” is meant regions of the nervous system other than the brain and spinal cord. For example, the peripheral nervous system includes sensory and motor nerve fibers that conduct signals to the brain or spinal cord.

By “central nervous system” is meant the brain or spinal cord, including the cerebrospinal fluid.

The present invention provides a number of advantages related to the treatment or prevention of pain. For example, administering inhibitors of IL-1β activity to the central nervous system of a mammal produces a greater decrease in pain than administrating these inhibitors to the peripheral nervous system. In addition, intrathecal administration of a compound that inhibits the production of the active form of IL-1β is as effective as a selective Cox-2 inhibitor in reducing mechanical and thermal pain sensitivity. Thus, administration of compounds that inhibit IL-1β activity in the central nervous system may be more effective than current methods for treating or preventing pain. In addition, smaller or less frequent doses of central IL-1β inhibitors may be required to achieve a therapeutic amount of these compounds. This use of smaller doses may minimize the frequency and severity of adverse side effects from these compounds.

Other features and advantages of the invention will be apparent from the following detailed description and from the claims.

### BRIEF DESCRIPTION OF THE DRAWINGS

FIGS. 1A and 1B are photographs of gels showing the induction of Cox-2 mRNA at 0, 2, 4, 6, 12, and 24 hours after unilateral hindpaw inflammation. Increased Cox-2 mRNA was detected in ipsilateral and contralateral rat lumbar L4/L5 spinal cord, inflamed skin, cervical spinal cord, and thalamus after intraplantar administration of complete Freund's adjuvant (CFA). FIG. 1C is a graph of the induction of Cox-2 mRNA in the ipsilateral and contralateral lumbar spinal cord based on the data in FIG. 1A. FIG. 1D is a graph of the relative expression of Cox-2 mRNA in the hindpaw, lumbar spinal cord, cervical spinal cord, and thalamus based on the data in FIG. 1B.

FIGS. 2A-2D are photographs showing the immunohistochemistry analysis of Cox-2 expression in dorsal horn neurons after 12 hours in inflamed (FIGS. 2B, 2C, and 2D) and naive rats (FIG. 2A) (scale: 50 μm). FIG. 2D is a higher magnification of the deep dorsal horn area of an inflamed rat shown in FIG. 1B. Based on double labeling with the neuronal marker NeuN, almost all of the Cox-2 immuno-labeled cells are neuronal (FIG. 2C).

FIG. 3 is a photograph showing Cox-2 mRNA distribution in the spinal cord 12 hours after inflammation (scale: 100 μm).

FIG. 4 is a bar graph showing the increase in prostaglandin E₂ (PGE₂) levels in cerebrospinal fluid after CFA-induced inflammation (“*” denotes p<0.01).

FIG. 5A is a schematic illustration of the preparation of a transverse spinal cord slice with an attached L4 dorsal root. FIG. 5B is a series of photographs of gels showing Cox-2 mRNA induction two hours after Aβ-, Aδ- or C-fiber dorsal root electrical stimulation in vitro. “N” denotes no stimulation; “I” denotes ipsilateral simulation; and “C” denotes contralateral stimulation. FIG. 5C is a photograph of a gel showing that the increase in Cox-2 mRNA in vivo three hours following electrical stimulation of the sciatic nerve for 30 minutes at C-fiber intensity was less than the increase after intraplantar CFA inflammation. FIG. 5D is a photograph of a gel showing that sciatic nerve blockade by perineural bupivacaine treatment, which is sufficient to abolish afferent activity, failed to eliminate the CFA-induced increase in Cox-2 mRNA levels six hours after inflammation. The fold-induction listed is measured with respect to naive rats after normalizing for β-actin mRNA levels. FIG. 5E is a bar graph showing the increase in PGE₂ levels after six hours in the cerebrospinal fluid of CFA-treated rats (CFA, n=13) and of CFA-treated rats with a sciatic nerve blockade (Block/CFA, n=6) (“*” denotes p<0.01 when compared to naive rats; “#” denotes p<0.05 when compared to CFA-treated rats).

FIG. 6A is a graph showing the increase in IL-1β levels in inflamed, ipsilateral hindpaw skin compared to IL-1β levels in the contralateral hindpaw, measured by ELISA (Safieh-Garabedian et al., Br. J. Pharmacol., 115:1265, 1995). FIG. 6B is a bar graph showing IL-1β levels in the cerebrospinal fluid two and fours hours following intraplantar CFA hindpaw inflammation (“N.D” means not detected). FIG. 6C is a photograph showing immunolocalization of IL1 receptor type I in dorsal horn (scale: 100 μm). FIG. 6D is a series of photographs of gels showing that IL-1β, but not TNFα (10 ng/ml), increased Cox-2 mRNA expression in the spinal cord in vitro after three hours. Intrathecal (i.t.), and to a lesser extent intravenous (i.v.), IL-1β increased Cox-2 mRNA levels in the spinal cord in vivo.

FIGS. 7A and 7B are photographs of gels showing the level of Cox-2 mRNA six hours after intravenous or intrathecal administration of IL-1 receptor antagonist (IL-1ra), respectively, prior to intraplantar CFA injection. FIG. 7C is a photograph of a gel showing that 200 ng/ml of IL-1ra did not affect activity-evoked Cox-2 mRNA induction in an in vitro spinal cord slice. FIG. 7D is a bar graph showing the effect of intrathecal administration, 15 min prior to intraplantar CFA injection, of 6 μg of IL-1 receptor antagonist (“CFA/IL-1ra”) or 1 nmole of caspase-1 inhibitor, Tyr-Val-Ala-Asp (“CFA/YVAD”), on Cox-2 mRNA expression six hours later (“*” denotes p<0.005 when compared to CFA, n=3). FIG. 7E is a bar graph showing that intrathecal administration of YVAD decreased PGE₂ levels in the cerebrospinal fluid by 50% at 12 hours post inflammation and by 70% at 24 hours post-inflammation (“*” denotes p<0.01 when compared to CFA, n=7 to 10 per group).

FIGS. 8A and 8B are graphs showing that intrathecal, but not intravenous, administration of the selective Cox-2 inhibitor NS398 (30 μg, obtained from RBI) 48 hours after induction of CFA inflammation decreased the mechanical (FIGS. 8A and 8B) and heat responses (FIG. 8D) of the treated rat towards the response levels observed prior to inflammation. In contrast, NS398 did not alter sensitivity in naive rats. FIGS. 8E and 8C are graphs showing that intrathecal administration of YVAD (1 nmol) did not significantly affect the thermal withdrawal latency but increased the mechanical threshold of the CFA inflamed hindpaw compared with vehicle treatment (p<0.05).

FIGS. 9A and 9B are bar graphs showing the level of sPLA₂ and cPLA₂ activity, respectively, in fresh rat skin, spinal cord, and whole brain homogenates from naive and inflamed animals 12 hours post-inflammation. The PLA₂ activity of the indicated tissue homogenate (30 μg) is expressed as counts per minute (cpm) of [¹⁴C] arachidonic acid released from [¹⁴C]2-arachidonyl-phosphatidylcholine in 30 minutes (FIG. 9A) or from [¹⁴C]2-arachidonyl-phosphatidylethanolamine in 120 minutes (FIG. 9B) (“*” denotes p=0.006, and “**” denotes p<0.05 when compared to naive rats based on the Student's t-test, n=4 rats). FIG. 9C is a photograph of a gel showing the Western Blot analysis of cPLA₂ expression in a naive rat and in an inflamed rat, 12 hours post inflammation. β-Actin was used as a loading control.

FIG. 10 is a photograph of a gel showing COX-2 mRNA induction after IL-1β treatment of DRG cells in vitro for four hours in the absence or presence of MAP kinase inhibitors PD98059 (an ERK inhibitor) and SB203580 (a p38 inhibitor).

FIG. 11 is a photograph of a gel showing COX-2 mRNA levels in L4 DRG four hours after intrathecal administration of saline (Sal) or IL-1β (10 ng).

FIG. 12 is a photograph of Western blot analysis of the induction of ERK and p38 by IL-1β in DRG primary cultures using phospho-specific antibodies for ERK (“pERK”) and p38 (“p-p38”).

FIG. 13 is a bar graph of PGE₂ levels in the culture medium of DRG primary cultures after stimulation with IL-1β (10 ng/ml) for four hours in the presence or absence of MAP kinase inhibitors SB203580 and PD98059.

FIG. 14 is a photograph of a gel showing the induction of Cox-2 mRNA expression in the spinal cord of neuropathic pain rat models, including the Spared Nerve Injury model (“SNI”) and the Chronic Constriction Injury model (“CCI”). Cox-2 mRNA was also induced after complete transection of the sciatic nerve (“Axotomy”).

FIGS. 15A-15D demonstrate that inflammation induces persistent p38 activation in DRG neurons. FIGS. 15A and 15B are photographs of immunohistochemistry analysis demonstrating that p38 phosphorylation is increased in DRG neurons two days after CFA injection into a hindpaw (scale bar, 50 μm). FIG. 15C is a bar graph showing the time course of p38 phosphorylation after CFA administration measured by the percentage of p-p38 positive neurons in the DRG [(n=5), * P<0.05; ** P<0.01, compared to naive control]. FIG. 15D is a bar graph showing the number of neurons with a certain level of staining. This graph demonstrates an increase in the intensity of p-p38 immunostained neurons two days after inflammation. Three hundred neurons from three animals were measured for each condition.

FIGS. 16A-16E are bar graphs demonstrating that p38 inhibition alleviates the late phase of CFA-induced inflammatory heat hyperalgesia. FIG. 16A indicates that intrathecal infusion of the p38 inhibitor SB 203580 does not change the development of inflammation in CFA-injected paws, based on paw thickness. SB 203580 (1 μg/μl) and control vehicle (saline) were infused by an osmotic pump (0.5 μg/0.5 μl/hour) connected to a catheter intrathecally implanted before CFA injection. FIGS. 16B and 16C are graphs showing the result of treatment with SB 203580 as described for FIG. 16A prior to CFA administration. This pre-treatment reduced the late phase of inflammatory heat hyperalgesia at 24 and 48 hours after CFA injection (FIG. 16B). Mechanical allodynia was not decreased (FIG. 16C). Heat and mechanical sensitivity were measured by paw withdrawal latency and paw withdrawal threshold, respectively, and expressed as a percentage of pre-CFA baseline measurements of vehicle control [**, P<0.01, compared to vehicle control (n=8)]. FIGS. 16D and 16E are graphs showing the effect of administering SB 203580 after treatment with CFA. Intrathecal injection of SB 203580 (1 μg) 24 hours after CFA treatment produced a delayed inhibition of inflammatory heat hyperalgesia. Heat and mechanical sensitivity were tested at 0.5, 3, and 24 hours post inhibitor administration [* P<0.05, compared to vehicle (saline) control (n=8)].

FIGS. 17A-17E illustrate that p38 activation mediates the inflammation-induced VR1 upregulation in DRG neurons. FIG. 17A is a photograph of a gel from an RNase protection assay. This assay failed to detect an increase in VR1 mRNA levels after CFA-induced inflammation. “Fold” represents comparative levels over control after normalizing with an actin control. FIG. 17B is a photograph of a Western blot indicating an increased expression of VR1 protein after inflammation. This upregulation at two days was blocked by intrathecal injection of SB203580 (1 μg, twice a day for two days). “Fold” represents comparative levels over control after normalizing with a loading control. FIG. 17C is a bar graph quantifying the level of VR1 protein from FIG. 17B (**, P<0.01, compared to control; +, P<0.05, compared to CFA, n=5). FIG. 17D is a photograph of immunohistochemistry analysis confirming an increase in VR1 levels two days after inflammation. This induction of VR1 protein expression was inhibited by SB203580 delivered using an osmotic pump as described for FIG. 16A (scale bar, 50 μm). FIG. 17E is a bar graph of the percentage of VR1-positive neurons based on FIG. 17D (**, P<0.01, compared to control; ⁺⁺, P<0.01, compared to CFA, n=5).

FIGS. 18A-18D demonstrate that CFA induces a sustained activation of ERK. FIG. 18A is a photograph of a low magnification image showing induction of ERK phosphorylation in laminae I-IIo neurons of the ipsilateral spinal cord (indicated with an arrowhead) 10 minutes after CFA injection into a hindpaw (scale bar, 200 μm). FIG. 18B is a high magnification image of FIG. 18A showing ERK activation in the medial superficial dorsal horn of the ipsilateral spinal cord 10 minutes after CFA injection (scale bar, 50 μm). FIG. 18C is a bar graph of a time course of pERK induction after CFA administration measured by the number of pERK positive neurons in the superficial (I-IIo) layers of the ipsilateral dorsal horn. Data is represented as mean+SEM (n=3) FIG. 18D is a photograph of a Western blot showing increased ERK phosphorylation of both ERK1 (44 kD) and ERK2 (42 kD) in the ipsilateral (I) dorsal horn, compared to contralateral (C) side, 30 minutes and 6 hours after CFA injection. The lower panel indicates levels of total ERK 1 and ERK2, as loading controls. “Fold” represents comparative levels over the corresponding contralateral side after normalizing for loading.

FIGS. 19A-19C show that CFA induces prodynorphin upregulation in the dorsal horn. FIG. 19A is a photograph of a gel from an RNase protection assay revealing an increase in prodynorphin m-RNA in the ipsilateral dorsal horn 24 and 48 hours after CFA injection. “Fold” represents comparative levels over control after normalizing for loading. FIG. 19B is a photograph of in situ hybridization indicating an increased expression of prodynorphin mRNA in ipsilateral superficial and deep dorsal horn neurons 24 hours after CFA treatment (scale bar, 50 μm). FIG. 19C is a photograph showing increased number of prodynorphin immunoreactive neurons induced in the ipsilateral superficial and deep dorsal horn by CFA injection at 48 hours (scale bar, 50 μm).

FIGS. 20A-20C show that ERK activation regulates prodynorphin expression. FIG. 20A is a photograph of a gel showing partial suppression of the CFA-induced increase in prodynorphin mRNA in the dorsal horn at 24 hours by U0126 (1 μg, intrathecally injected 30 minutes before and 6 hours after CFA). “Fold” represents comparative levels over control after normalizing for loading. FIG. 20B is a bar graph showing the quantification of prodynorphin mRNA-positive neurons in laminae I-II and III-VI of the ipsilateral dorsal horn 24 hours after CFA injection [*, P<0.001, compared to control; +, P<0.001, compared to CFA (n=4)]. FIG. 20C is a photograph of in situ hybridization showing an inhibition of the CFA-induced increase in prodynorphin mRNA labeled neurons in the superficial dorsal horn by U0126 24 hours after CFA injection (Scale bar, 50 μm).

FIGS. 21A-21C demonstrate that ERK activation regulates NK-1 expression. FIG. 21A is a photograph showing suppression of the CFA-induced increase in NK-1 immunoreactivity in the medial superficial dorsal horn at 48 hours by U0126 delivered via an osmotic pump (scale bar 50 μm). FIG. 21B is a bar graph showing quantification of the numbers of NK-1 neurons in lamina I-IIo of the ipsilateral dorsal horn 48 hours post CFA injection [*, P<0.001, compared to control; ⁺, P<0.001, compared to CFA (n=5)]. FIG. 21C is a photograph of a Western blot indicating that the CFA-induced NK-1 increase in the dorsal horn at 24 hours is inhibited by U0126 (1 μg, intrathecally injected 30 minutes before and 6 hours after CFA injection). CREB, a constitutively expressed protein was used as a loading control. “Fold” represents comparative levels over control after normalizing for loading.

FIGS. 22A-22F demonstrate that ERK is activated in a subset of prodynorphin- and NK-1-expressing neurons. pERK is largely colocalized with prodynorphin (FIGS. 22A, 22C, and 22E) and NK-1 (FIGS. 22B, 22D, and 22F) in the medial superficial dorsal horn 24 hours after CFA injection. Arrows indicate double labeled neurons (scale bar, 20 μm).

FIGS. 23A and 23B are bar graphs illustrating that sustained infusion of a MEK inhibitor reduces CFA-induced inflammatory pain. The MEK inhibitor U0126 delivered by osmotic pump (0.5 μg/μl/hr) prior to CFA injection reduces thermal hyperalgesia (FIG. 23B) and mechanical allodynia (FIG. 23B) 24 and 48 hours after CFA injection. These were measured by paw withdrawal latency and paw withdrawal threshold, respectively, and expressed as a percentage of pre-CFA baseline measurements of vehicle control (50% DMSO) [* P<0.01, compared to vehicle control (n=8)].

FIGS. 24A and 24B are bar graphs showing that post-treatment with a MEK inhibitor has a delayed effect on inflammatory pain. U0126 (1 μg) or vehicle (10% DMSO) was intrathecally administered 24 hours after CFA injection. Heat hyperalgesia (FIG. 24A) and mechanical allodynia (FIG. 24B) were tested 30 minutes, 6 hours, and 24 hours after the administration of the U0126 [* P<0.05, compared to corresponding vehicle control (n=10)]. The data is expressed as a percentage of pre-CFA baseline measurements of vehicle control.

FIG. 25 is a schematic illustration of pathways involved in pain hypersensitivity.

FIGS. 26A-26F demonstrate that NGF is required for p38 activation following inflammation and results in VR1 upregulation via p38 in the DRG. FIGS. 26A and 26B are bar graphs illustrating that NGF antiserum treatment (i.p., 84 mg/g body weight, once a day for 2 days) not only alleviates inflammatory pain, especially heat hyperalgesia, but also suppresses CFA-induced increase in p-p38 and VR1 levels in the DRG (*, P<0.05; **, P<0.01; compared to CFA, n=3). FIGS. 26C and 26D are a photograph and a bar graph showing that intrathecal injection of NGF (2 μg, twice a day for 3 days) increases p-p38 and VR1 expression (**, P<0.01; compared to control (saline), n=3, scale bar, 50 μm). FIG. 26E is a bar graph showing that the NGF-induced VR1 increase is blocked by p38 inhibitor 203580 (1 μg) coadministered with NGF for 3 days [**, P<0.01, compared to vehicle (saline); ⁺⁺, P<0.01, compared to NGF, n=3]. FIG. 26F is a bar graph of intensity frequency showing that the increase in the intensity of VR1 immunostained neurons after intrathecal NGF is also decreased by p38 inhibition. Three hundred neurons from 3 animals were measured for each condition.

### DETAILED DESCRIPTION

The present methods stem from the discovery that a significant factor for the induction of Cox-2 expression and prostanoid production in the central nervous system in response to peripheral inflammation is upregulation of IL-1β in the central nervous system. In contrast, sensory inflow from nerve fibers innervating the site of peripheral inflammation produced a much smaller increase in central Cox-2 levels. Additionally, production of prostanoids in the central nervous system which contribute to pain sensitivity was correlated with the level of central Cox-2 rather than the level of an upstream enzyme, phospholipase₂.

Induction of Cox-2 in the central nervous system and the resulting increase in central prostanoid production may contribute to primary hyperalgesia, secondary hyperalgesia, and diffuse pain. For example, central Cox-2 may participate in the establishment and maintenance of chronic peripheral inflammatory hypersensitivity by facilitating transmitter release from nociceptor fibers, as well as through direct activation of prostanoid receptors in the central nervous system. The heightened sensitivity adjacent to inflamed tissue is probably mediated by central prostanoids and by the neuromodulators which are released from the central terminals of sensory fibers innervating the inflamed tissue. The diffuse aches and pains typical of many inflammatory diseases and the fever, lethargy, and anorexia associated with infectious diseases may also be caused by the widespread induction of Cox-2 and ensuing prostanoid production in the central nervous system after peripheral inflammation.

Administration of an inhibitor of IL-10 production to the rat spinal cord reduced the increases in central Cox-2 mRNA levels, central prostaglandin₂ levels, and mechanical and thermal pain sensitivity that were induced by peripheral inflammation. Thus, inhibition of IL-1β activity in the central nervous system is a useful means for preventing, reducing, or stabilizing pain in mammals, such as humans.

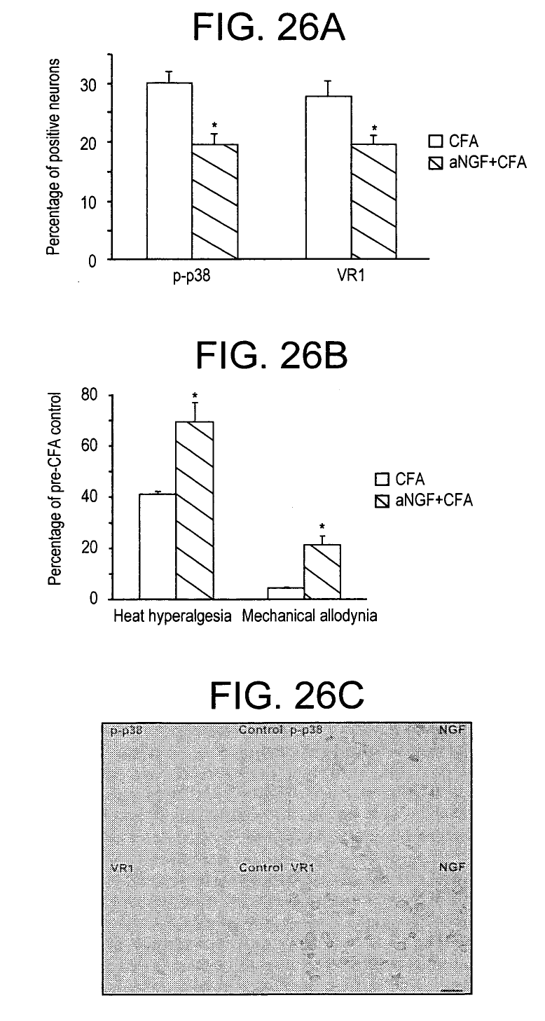

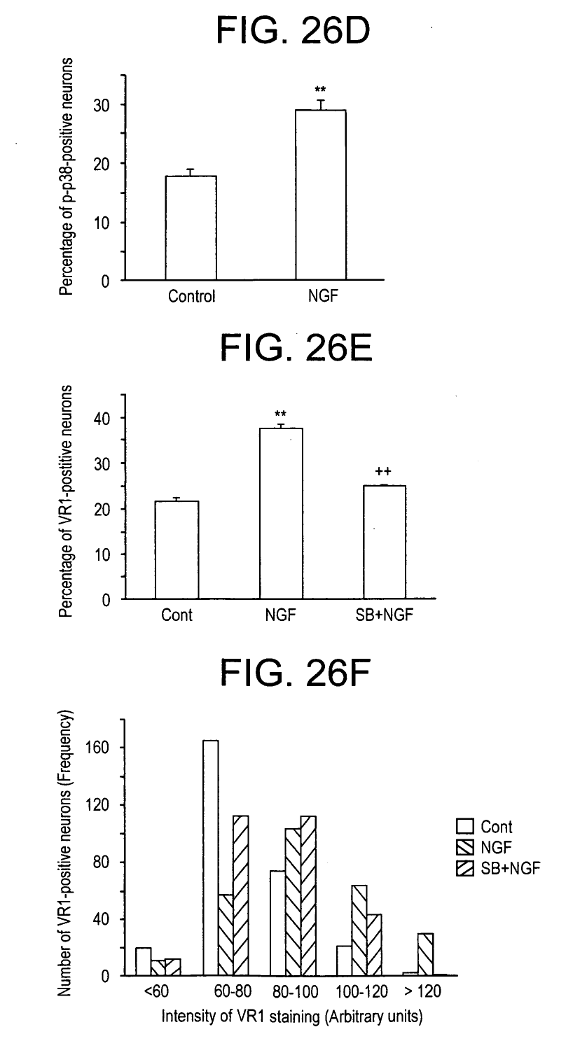

We also discovered that peripheral inflammation activates the MAP kinase p38 in primary sensory neurons, but not in second order dorsal horn neurons. In particular, p38 was predominantly activated in C-fiber nociceptors in dorsal root ganglion (DRG) neurons after inflammation. Intrathecal delivery of a p38 inhibitor alleviated inflammatory heat but not mechanical hyperalgesia. The VR1 receptor, which mediates heat hypersensitivity, is a target of p38 activation. VR1 protein, but not VR1 mRNA, was upregulated by inflammation, and p38 was primarily activated in VR1-expressing neurons. p38 activation was required for the inflammation-evoked increase in VR1 protein levels. Nerve growth factor (NGF, a know pain mediator) may be the trigger for these events. NGF is required for inflammation-induced p38 activation and VR1 upregulation, and the NGF-induced upregulation of VR1 is mediated by p38 (FIGS. 26A-26F).

Activation of another MAP kinase, ERK, has two roles in nociceptive plasticity in the dorsal horn: a short-latency contribution to acute noxious stimulus-induced central sensitization and an involvement in the induction and maintenance of inflammatory pain. pERK's involvement in peripheral inflammatory pain hypersensitivity may result from, at least in part, its regulation of the expression of the pain mediators prodynorphin and NK-1 and other target genes. ERK activation plays, therefore, a pivotal role in the functional plasticity and chemical phenotype of a group of neurons in the superficial dorsal horn, determining the activation of particular effector responses to divergent inputs, which in turn contribute to altered sensibility.

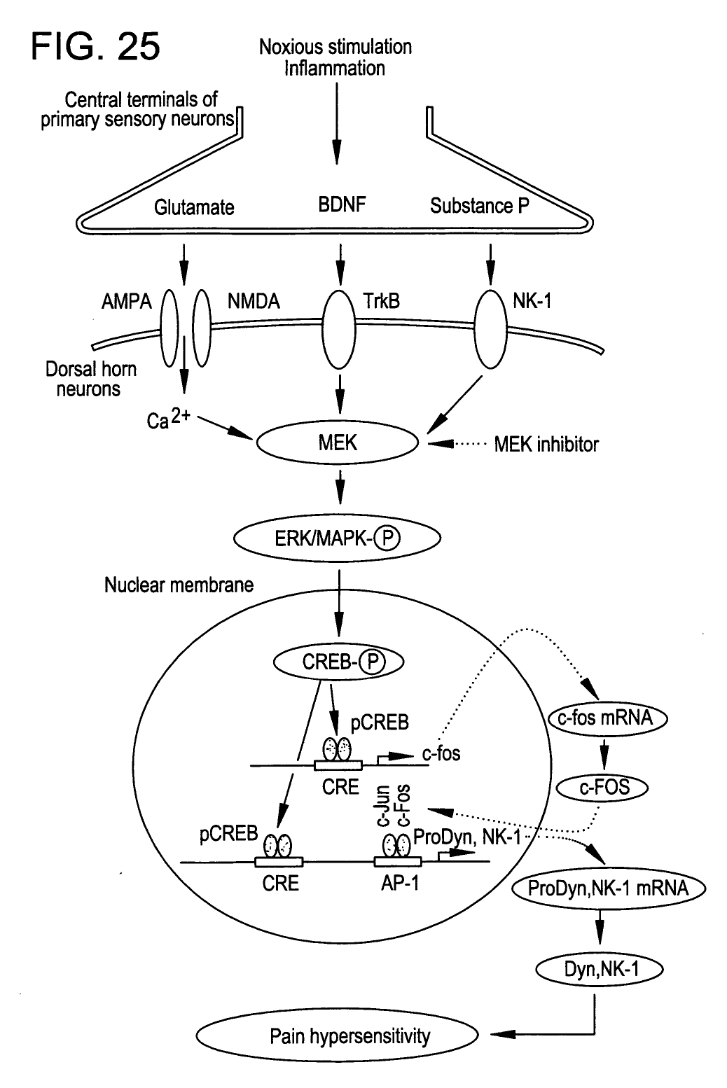

Administration of IL-1β to primary sensory neurons also increased the phosphorylation of the MAP kinases p38 or ERK (FIG. 25). Inhibitors of these kinases reduced the increase in Cox-2 mRNA and PGE₂ induced by IL-1β. These results support the ability of inhibitors of MAP kinase phosphorylation and/or activity to reduce or prevent pain.

The experiments described above were carried out as follows.

### EXAMPLE 1

### Central Induction of Cox-2

The extent of Cox-2 induction in the central nervous system following unilateral hindpaw inflammation evoked by complete Freund's adjuvant (CFA) was investigated. CFA (100 μl, Sigma) was injected into the left rat hindpaw of rats anaesthetized with 2% halothane. The level of Cox-2 mRNA was measured using RNase protection assays which were performed on total RNA extracts from spinal cord samples using the RPA III kit (Ambion). The template for a Cox-2 radiolabeled riboprobe was generated by PCR from rat dorsal root ganglia cDNA using primers 5′-GCAAATCCTTGCTGTTCCAACCCA-3′ (SEQ ID NO: 1) and 5′-TTGGGGATCCGGGATGAACTCTCT-3′ (SEQ ID NO: 2) and subsequently cloned into pCRII (Invitrogen). At least two assays were used for each observation, and the fold change in Cox-2 mRNA levels was determined with respect to a β-actin loading control.

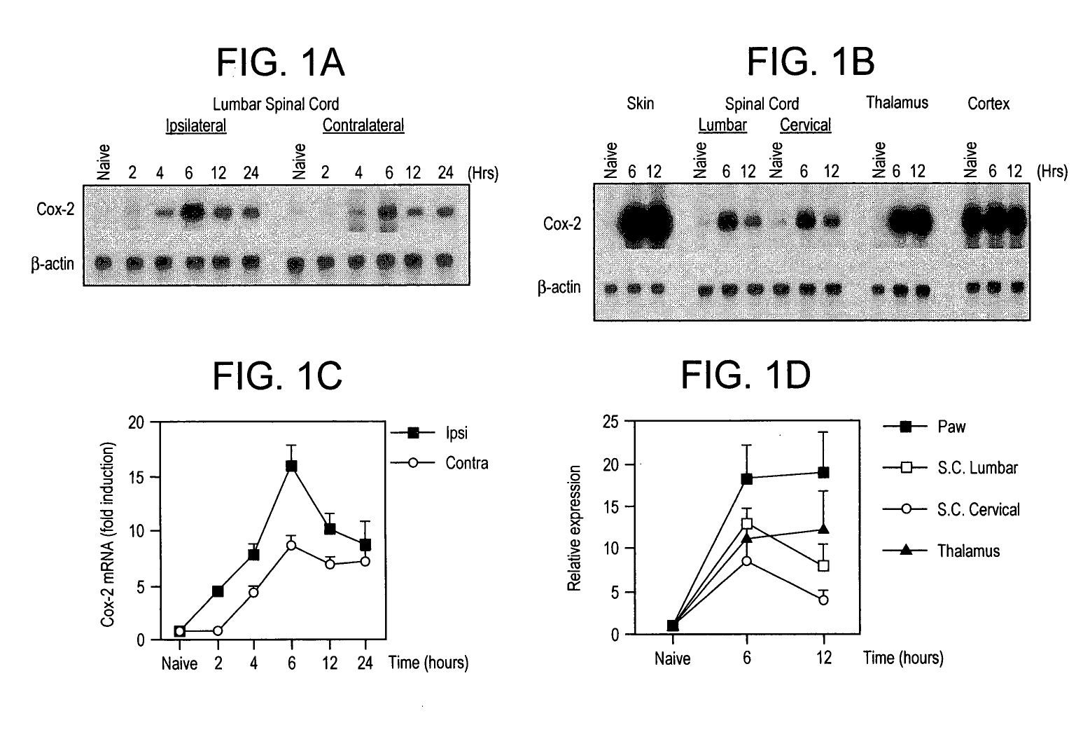

Local peripheral inflammation produced a rapid, time-dependent, increase in Cox-2 mRNA levels in the region of the lumbar spinal cord where paw afferents terminate (L4/L5 segments) (FIG. 1A). Maximal induction occurred at six hours (16-fold) and persisted for at least 24 hours (9-fold). At early times (two to six hours), the increase was greatest ipsilateral to the inflammation. Unexpectedly, by 12 and 24 hours, the levels were bilaterally symmetrical in the lumbar spinal cord (FIGS. 1A and 1C), suggesting a generalized increase of Cox-2 expression.

To confirm this possible generalized increase in Cox-2 expression, levels of Cox-2 were measured in multiple regions of the rat. Hindpaw inflammation produced an increase in Cox-2 expression in the skin (18-fold) and at all levels of the spinal cord (FIG. 1B). Increases in Cox-2 expression were also observed in the pons, ventral midbrain, hypothalamus, and thalamus (FIGS. 1B and 1D). Cox-2 expression in the cerebral cortex is constitutive and was not significantly affected by inflammation (FIG. 1B).

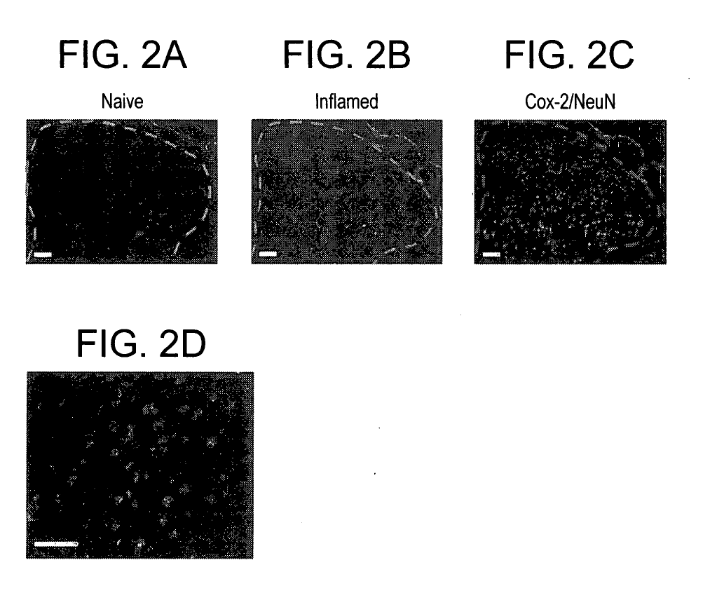

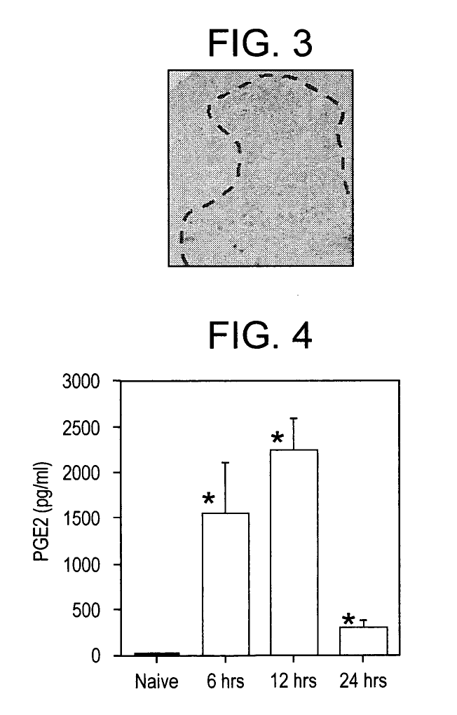

To visualize the induction of Cox-2 protein in dorsal horns after peripheral inflammation, immunohistochemistry was performed on transverse spinal cord sections (20 μm) using a Cox-2 antibody (Santa Cruz) and a neuronal marker, the NeuN antibody (Chemicon), as described previously (Amay et al., Mol. Cell Neurosci. 15:331-342, 2000). In situ hybridization was performed using a digoxygenin-labeled riboprobe to visualize the induction of Cox-2 mRNA (Mannion et al., Proc. Natl. Acad. Sci. U. S. A. 96:9385, 1999). Low basal levels of Cox-2 mRNA and protein were present in dorsal horn neurons with a substantial upregulation six and 12 hours post-inflammation (FIGS. 2A-2D and 3). To determine whether this induction of Cox-2 was correlated with an increase in PGE₂ levels, a 100-200 μl sample of cerebrospinal fluid was withdrawn from the cisterna magna of anaesthetized rats using a 27 G needle, and PGE₂ levels were analyzed using a PGE₂ enzyme-immunoassay according to the manufacturer's protocol (Amersham Pharmacia Biotech). Indeed, induction of Cox-2 was associated with a greater than 80-fold increase in PGE₂ levels in the cerebrospinal fluid, which peaked at 12 hours post-inflammation (FIG. 4).

Cox-2 expression is also induced in the central nervous system by nerve injury. In particular, Cox-2 mRNA was induced in the spinal cord of neuropathic pain rat models, such as the Spared Nerve Injury model (“SNI,” Decosterd and Woolf, Pain 87:149-158, 2000) and the Chronic Constriction Injury model (“CCI,” Bennett and Xie, Pain 33:87-107, 1988) (FIG. 14). Cox-2 mRNA was also induced after complete transection of the sciatic nerve (“Axotomy”). Thus, in addition to their role in inflammatory pain, central prostanoids also contribute to neuropathic pain, which is a form of pathological pain induced by nerve injury.

### EXAMPLE 2

### Effect of Sensory Inflow on Central Cox-2 Levels

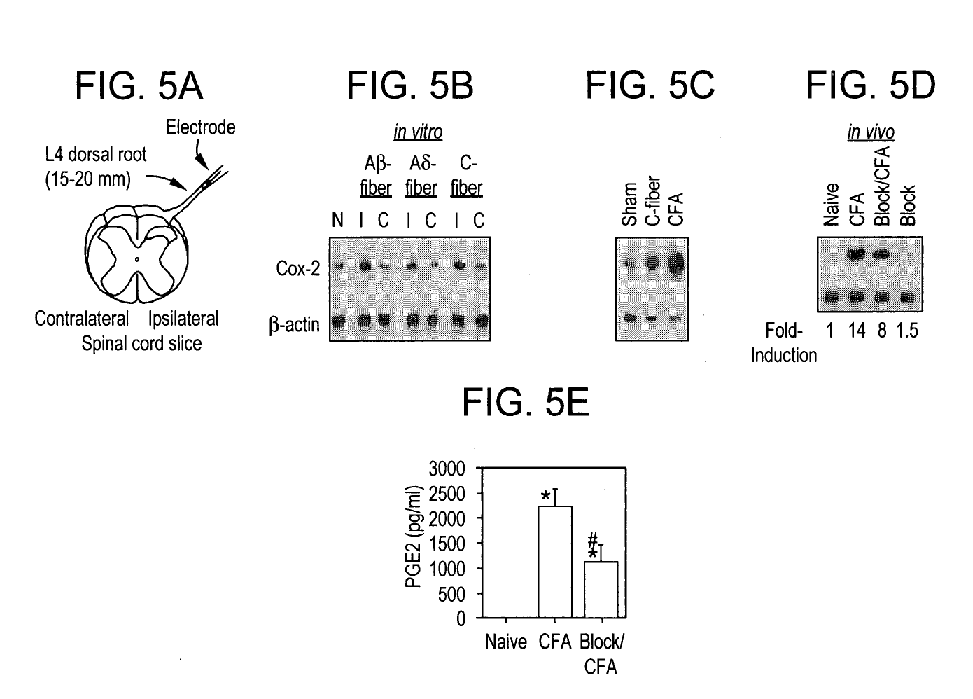

Two mechanisms may contribute to central Cox-2 induction in response to peripheral inflammation: 1) sensory inflow from nerve fibers innervating the inflamed hindpaw or 2) circulating pro-inflammatory cytokines. To determine whether primary sensory neuron activity increases central Cox-2, an isolated adult rat spinal cord slice preparation with an attached dorsal root was analyzed (FIG. 5A). To prepare this spinal cord slice, the lumbar spinal cord from an adult rat under urethane anesthesia was removed and immersed in cold Kreb's solution. A 700-μm-thick transverse slice with attached L4 dorsal root (15-20 mm) was prepared and perfused with Kreb's solution saturated with 95% O₂ and 5% CO₂ at 36-37° C. (Baba et al., J Neurosci 19:859, 1999). The L4 dorsal root was electrically stimulated for 30 minutes using a suction electrode at 20 μA (0.05 ms, 50 Hz) for Aβ-fibers, 100 μA (0.05 ms, 50 Hz) for Aβ-fibers, and 1000 μA (0.5 ms, 50 Hz) for C-fibers. The slices were perfused with Kreb's solution for two hours before electrical stimulation, and for three hours after stimulation.

Electrical stimulation of the dorsal root at Aβ-, Aδ-, or C-fiber intensity for 30 minutes increased Cox-2 mRNA after three hours in the ipsilateral dorsal horn by two to three-fold compared with the contralateral dorsal horn or non-stimulated slices (FIG. 5B). Sciatic nerve stimulation at C-fiber intensity for 30 minutes in vivo, which was performed as previously described (Mannion et al., Proc. Natl. Acad. Sci. U. S. A. 96:9385, 1999), also induced Cox-2 expression in the lumbar spinal cord three hours later. However, this three to four-fold increase in Cox-2 expression was less than the over 10-fold increase that followed peripheral inflammation (FIG. 5C).

To determine if sensory inflow from nerve fibers innervating the inflamed hindpaw was sufficient to explain the large induction of central Cox-2 following peripheral inflammation, an injectable suspension of biodegradable bupivacaine-polyester microspheres was used to produce a nerve block 30 minutes before injection of CFA into rat hindpaws (n=6) (Curley et al., Anesthesia 84:1401, 1996). Based on behavior assessments and previously reported electrophysiological studies, this nerve block resulted in complete sensory and motor blockade of the sciatic nerve for 48 hours. This blockage of conduction in the sciatic nerve reduced, but did not eliminate, PGE₂ levels in the cerebrospinal fluid (FIG. 5E) and Cox-2 mRNA induction in the lumbar ipsilateral spinal cord after hindpaw inflammation (5±1.5 fold, n=3, FIG. 5D). Therefore, although sensory inflow is sufficient to induce central Cox-2, an additional factor must contribute to central Cox-2 induction following peripheral inflammation.

### EXAMPLE 3

### Upregulation of IL-1β and its Effect on Central Cox-2 Levels

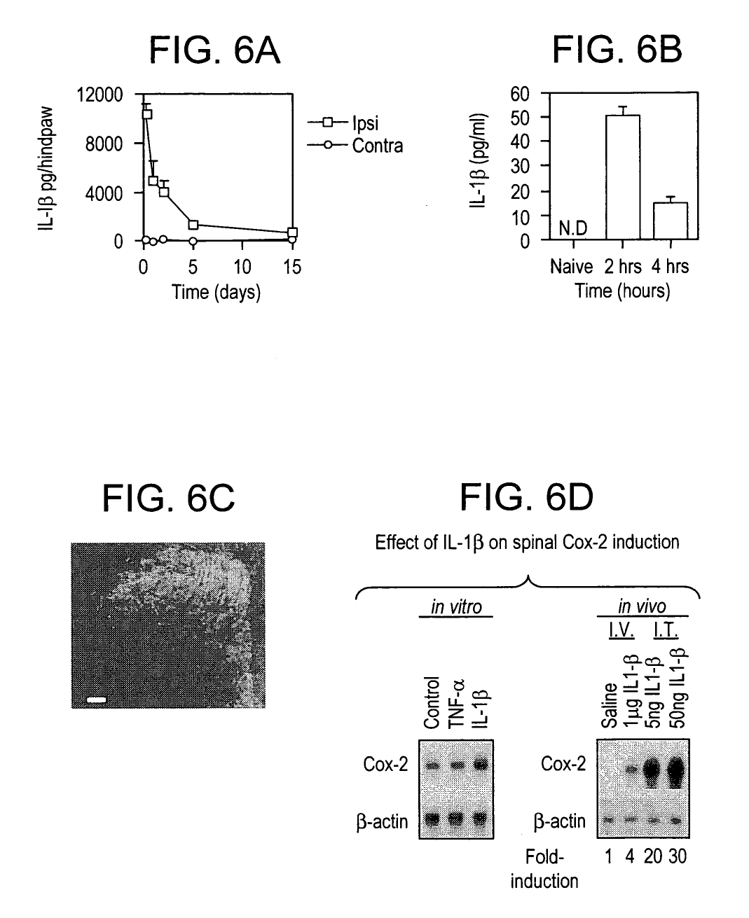

To determine whether IL-1β also contributes to the induction of central Cox-2, IL-1β levels were measured in the peripheral and central nervous systems of the rat using ELISA as previously described (Safieh-Garabedian et al., Br. J. Pharmacol. 115:1265-1275, 1995). A massive upregulation of IL-1β (>10,000-fold) occurred in the inflamed paw soon after CFA administration and lasted for several days (FIG. 6A). IL-1β also showed 50 and 20-fold increases in the cerebrospinal fluid, two and four hours post-inflammation, respectively (FIG. 6B), which preceded the peak upregulation of Cox-2 mRNA. Based on immunohistochemistry with an anti-IL-1 receptor type I antibody (Research Diagnostics) that was performed as described previously (Amaya et al., supra), the type-I IL-1β receptor was heavily expressed in the spinal cord, especially in laminae I-III of the dorsal horn (FIG. 6C), an area where Cox-2 is induced after inflammation.

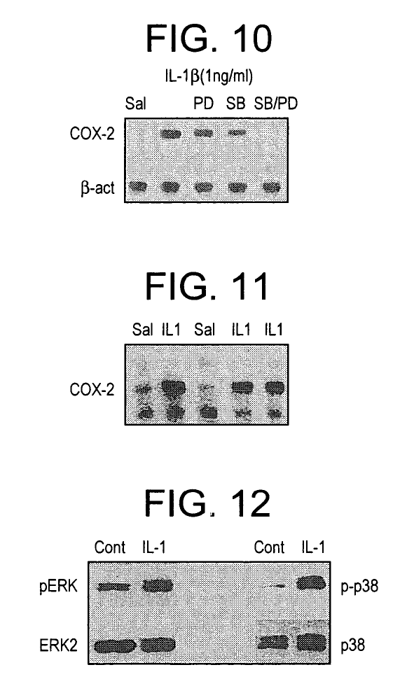

To determine whether IL-1β induces central Cox-2, rats were subjected to intravenous or intrathecal administration of this cytokine, and Cox-2 mRNA levels in the lumbar spinal cord were assessed five hours later. Intravenous IL-1β (1 μg) upregulated Cox-2 mRNA four-fold in the spinal cord (FIG. 6D) but a much greater effect was produced by intrathecal injection of 5 or 50 ng IL-1β, (20- or 30-fold, respectively) (FIG. 6D). IL-1β also induces Cox-2 expression in dorsal root ganglion neurons (“DRG”), which are primary sensory neurons, both in vivo after intrathecal IL-1β administration and in vitro in primary neuronal cultures. In particular, Cox-2 mRNA and protein levels increased significantly in cultured DRG neurons after administration of 1 or 10 ng/ml IL-1β. For this analysis, Cox-2 mRNA levels were monitored using an RNase protection assay, and protein expression was visualized by immunohistochemistry, as described herein (FIG. 10). Intrathecal administration of IL-1β (1 and 10 ng) also substantially upregulated Cox-2 mRNA levels in rat lumbar DRG neurons four hours after treatment (FIG. 11). Moreover, central PGE₂ levels were significantly upregulated (50-fold) following intrathecal administration of IL-1β (5 ng) to 1204±360 pg/ml from naive levels of 24±10 pg/ml (p<0.005, n=6). This result suggests that the central IL-1β mediated induction of Cox-2 leads to central prostanoid production.

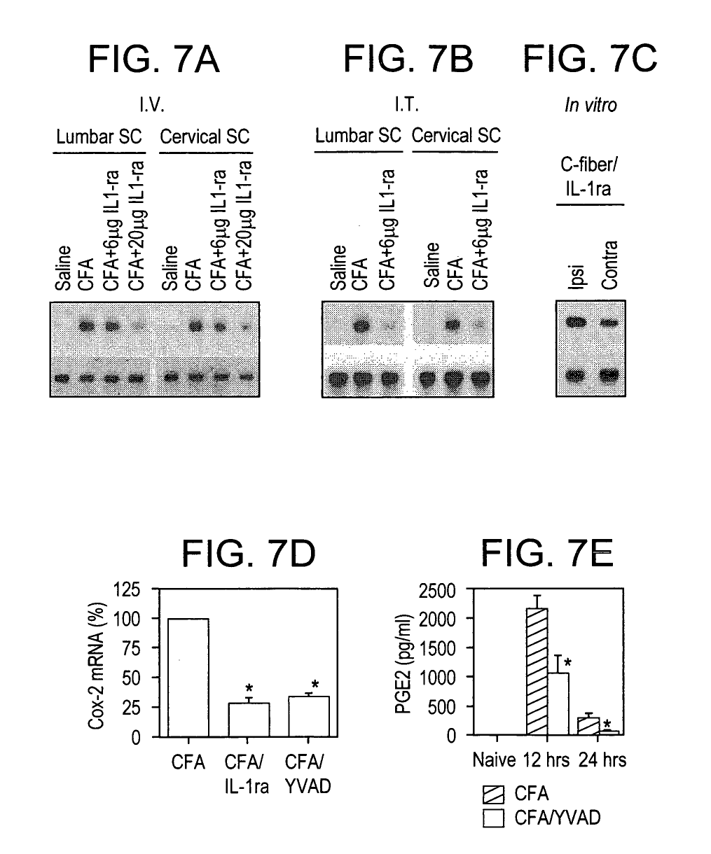

To further support the role of central IL-1β in induction of Cox-2, the effect of inhibiting IL-1β activity was determined. Central IL-1β activity was inhibited by intrathecal administration of either a recombinant IL-1β receptor antagonist (IL-1ra) or an inhibitor of caspase-1 (also known as interleukin-1β converting enzyme which converts pro-interleukin-1β to the active form of IL-1β). Intrathecal administration of 6 μg of IL-1ra 30 minutes prior to CFA injection reduced Cox-2 mRNA levels by 75% six hours post-inflammation (FIG. 7B). A 30 minute intrathecal pretreatment with the caspase-1 inhibitor, YVAD (acetyl-Tyr-Val-Ala-Asp-CHO from Calbiochem, 1 nmole, 0.5 μg) blocked the induction of spinal Cox-2 mRNA six hours after peripheral inflammation by 65% (FIG. 7D). Intrathecal pretreatment with YVAD (1 nmole) also reduced the levels of central PGE₂ at 12 and 24 hours post-inflammation by 50% (FIG. 7E). These data show that IL-1β in the spinal cord is responsible, perhaps acting together with other cytokines, for central transcriptional activation of Cox-2 after peripheral inflammation, and that Cox-2 induction is the major limiting factor in the production/release of central PGE₂.

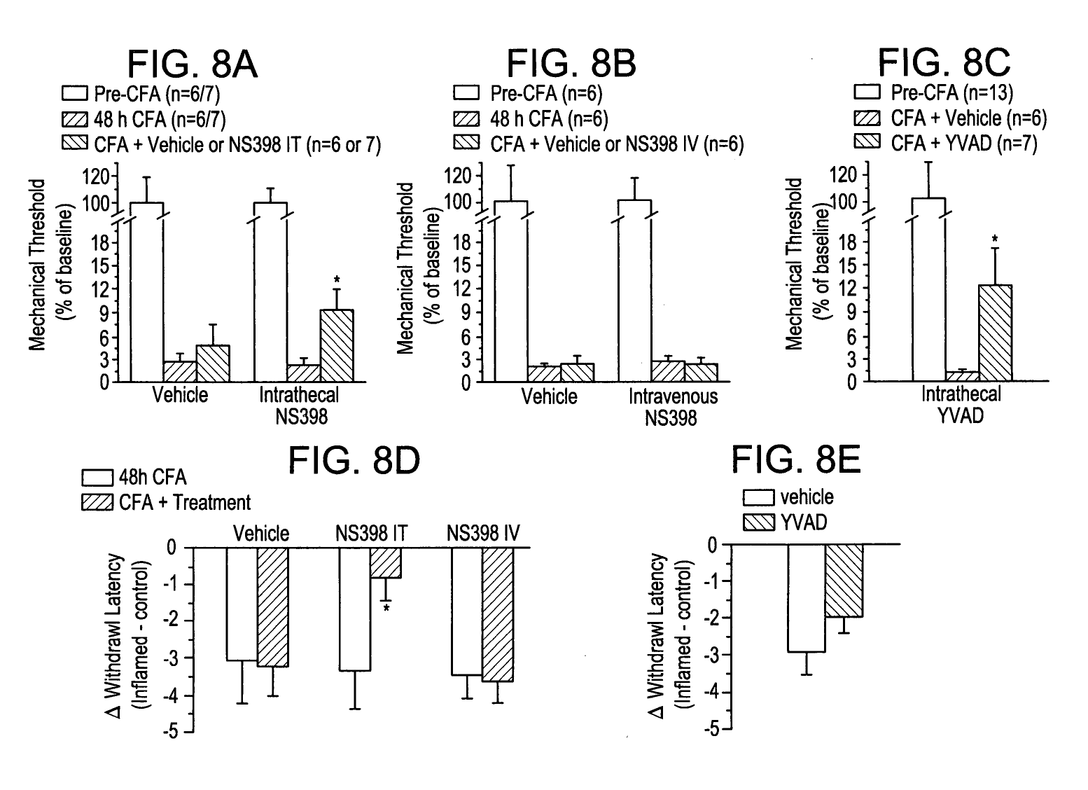

To test the behavioral consequences of central Cox-2 induction, the effect of administrating the caspase-1 inhibitor, YVAD, or the Cox-2 inhibitor, NS398, on mechanical and thermal sensitivity in rats was determined. Mechanical hypersensitivity was assessed in naive and inflamed rats using calibrated von Frey filaments (0.017-95.5) as described previously (Decosterd et al., Pain 87:149-158, 2000). Thermal sensitivity was assessed by applying a beam of radiant heat and recording the paw withdrawal latency (Decosterd et al., supra). Intrathecal, but not intravenous, administration of NS398 (30 μg) significantly reduced both mechanical (4.2-fold) and thermal hyperalgesia 48 hours after peripheral CFA-induced inflammation (FIGS. 8A, 8B, and 8D). Intrathecal administration of YVAD (1 nmol) had an even greater effect on normalizing mechanical sensitivity (12-fold) (FIG. 4D). Both the Cox-2 and caspase-1 inhibitors had no significant effect on mechanical or thermal pain sensitivity in naive rats. These results suggest that compounds which inhibit IL-1β activity in the central nervous system are useful in the treatment and prevention of pain in mammals.

### EXAMPLE 4

### Role of MAP Kinases and NFκ-B in Induction of Cox-2

To investigate the intracellular signaling pathway that leads to the increase in Cox-2 mRNA in the DRG neurons following treatment with IL-1β, the activation of the NFκ-B transcription factor and members of the MAP kinase family in primary DRG neurons was studied. Administration of IL-1β to DRG cell cultures lead to phosphorylation of p38 and ERK. Phosphorylated p38 and ERK were visualized by standard Western blot analysis and immunohistochemistry 30 minutes after administration of 10 ng/ml IL-1β to the culture medium (FIG. 12). The expression of the specific IL-1 receptor type I on the DRG neurons suggests that these members of the MAP kinase family are activated by the direct action of IL-1β on primary sensory neurons.

To measure the contribution of p38, ERK, and NFκ-B to IL-1β-mediated induction of Cox-2 mRNA and protein levels, specific inhibitors for each of these factors were used. p38 and ERK inhibitors each decreased the induction by IL-1β of Cox-2 mRNA in the primary DRG neuron cultures by 30% (FIG. 10). In contrast, a NFκ-B specific inhibitor did not affect the increase in Cox-2 mRNA levels that is induced by IL-1β treatment. Combining both p38 and ERK MAP kinase inhibitors led to a dramatic decrease in the induction of Cox-2 mRNA by IL-1β, indicating that p38 and ERK act synergistically to upregulate Cox-2 mRNA in response to the IL-1β treatment (FIG. 10).

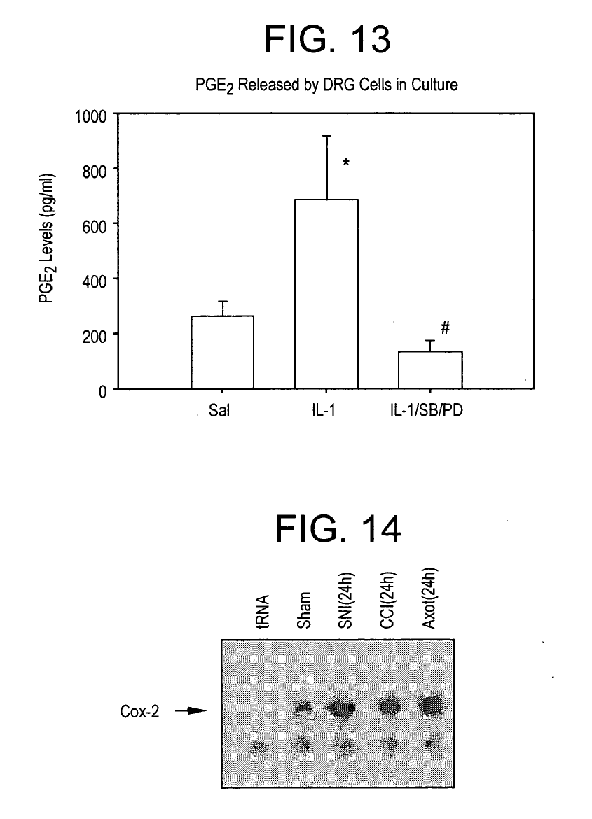

In order to assess the functional relevance of COX-2 upregulation as well as its blockade by MAP kinase inhibitors, levels of PGE₂ secreted in vitro by the cultured DRG neurons were quantified by ELISA after IL-1β treatment. The upregulation of Cox-2 mRNA and protein levels in primary sensory neurons lead to an increase in PGE₂ synthesis. This increase in PGE₂ levels was reduced by the MAP kinase inhibitors (FIG. 13).

These results indicate that the signaling mechanisms responsible for the induction of Cox-2 expression in neurons and prostanoid release include members of the MAP kinase family. The ability of MAP kinase inhibitors to block both the induction of Cox-2 by IL-1β and the subsequent release of prostanoids make these inhibitors useful candidate analgesics for reducing peripheral and central prostanoid release and alleviating pain hypersensitivity.

### EXAMPLE 5

### Characterization of the Role of P38 MAP Kinase in Pain Hypersensitivity

We discovered a novel role for p38 MAP kinase in producing inflammatory heat pain hypersensitivity acting on dorsal root ganglion (DRG) neurons in the peripheral nervous system. Injection of complete Freund's adjuvant (CFA) into a rat hindpaw produced sustained inflammation and a persistent activation of p38 in C-fiber nociceptor primary sensory neurons in DRG neurons. In contrast to ERK activation, no increase in p38 activation was found in the dorsal horn after inflammation. Inflammation also produced a sustained upregulation of vanilloid receptor subtype 1 (VR1) protein, but not mRNA, in DRG neurons. VRI is the receptor responsible for detecting noxious heat stimuli and also mediates inflammatory heat hyperalgesia. Intrathecal infusion of the p38 inhibitor SB203580 blocked the inflammation-induced increase in VR1 protein levels and alleviated the maintenance of inflammation-induced heat hyperalgesia. In contrast, the inhibitor did not significantly affect mechanical allodynia, the severity of the inflammation (i.e., swelling), or basal pain sensitivity (i.e., pain in the absence of inflammation). The activation of p38 by inflammation required nerve growth factor (NGF), which is produced in inflamed tissues and promotes inflammatory pain by driving peripheral sensitization and by increasing gene expression in sensory neurons. Conversely, p38 activation was required for NGF-induced VR1 upregulation (FIGS. 26A-26F). Thus, p38 activation in C-fiber nociceptors in the peripheral nervous system likely contributes to inflammatory heat hyperalgesia by increasing VR1 translation in a NGF-dependent way.

The experiments are described in more detail below.

p38 Activation in the DRG

To assess whether localized peripheral inflammation leads to p38 activation in DRG neurons, 100 μl of CFA was injected into the plantar surface of the left hindpaw of adult male Sprague-Dawley rats (240-320 g), maintained according to Massachusetts General Hospital Animal Care institutional guidelines and placed under pentobarbital anesthesia (50-60 mg/kg, i.p.). This CFA injection induced a localized inflammation that developed over several minutes, lasted more than a week, and manifested as swelling, erythema, and inflammatory pain (Stein et al., Pharmacol Biochem Behav 31: 455-51, 1988; Safieh-Garabedian et al., Br. J. Pharmacol. 115:1265, 1995).

To determine whether the phosphorylation of p38 was induced by this CFA treatment, immunohistochemistry was performed. Immunostaining of perfusion-fixed tissue was used to quantify p-p38 levels because fixatives stop phosphorylation immediately. For fresh tissue, mechanical dissection procedures, such as cutting axons and cooling down tissue during DRG collection, are strong sensory stimuli and thus would cause changes in the phsophorylation levels of sensory neurons. Therefore, Western analysis was not used to examine p38 phosphorylation in DRG neurons.

For this immunohistochemistry analysis, rats were perfused through the ascending aorta with saline followed by 4% paraformaldehyde with 1.5% picric acid. L4 and L5 DRGs and L4-L5 spinal cord segments were dissected. DRG and transverse spinal cord sections (20 μm) were cut and processed for immunohistochemistry using the previously described ABC method (Ji et al., Nat. Neurosci. 2:1114-1119, 1999), with polyclonal anti-p38 and phospho-p38 (1:300, New England BioLabs) and polyclonal anti-VR1 antibodies (1:5000, Kindly provided by Glaxso Welcome and Dr. David Julius). For colocalization studies, immunofluorescence was performed to analyze the double staining between antibodies reactive with phosphop38 (anti-rabbit, 1:300) and VR1 (anti-guinea pig, 1:3000, Neuromics) or P2X3 (anti-guinea pig, 1:3000, Neuromics) or NeuN (anti-mouse, 1:3000, Chemicon). This double immunofluorescence was performed as previously described (Ji et al., 2001, supra). Briefly, DRG and spinal sections were incubated with a mixture of two primary antibodies overnight at 4° C. and then incubated with a mixture of FITC- and CY3-congugated secondary antibodies (1:300, Jackson immunolab) for two hours at room temperature. A Tyramide Signal Amplification (TSA, NEN) kit was used to perform double staining with two polyclonal rabbit antibodies (phospho p38 and TrkA) as previously reported (Amaya et al., Mol. Cell Neurosci 15:331-342, 2000). The images of immunostained DRG sections were captured with a CCD camera. The intensity of immunostaining was measured with computer-assisted software (IP lab), and the number of immunoreactive neuronal profiles was counted in a blinded fashion. This counting did not determine the total numbers of cells (Coggeshall and Lekan, 1996). Instead, the results provide a way to evaluate differences in staining levels between control and treated animals. To prevent variations due to experimental conditions, the control and treated DRG sections were mounted on the same slides and processed under the same conditions. The threshold to pick up a positive cell was set at a level that did not detect weakly stained cells. As previously reported (Ji et al., 1999, supra), every fifth section was picked from a series of consecutive DRG sections (20 μm), and four sections were included for each DRG. The percentage of immnunoreactive neuronal profiles=(number of positive stained neuronal profiles/number of total neuronal profiles)×100.

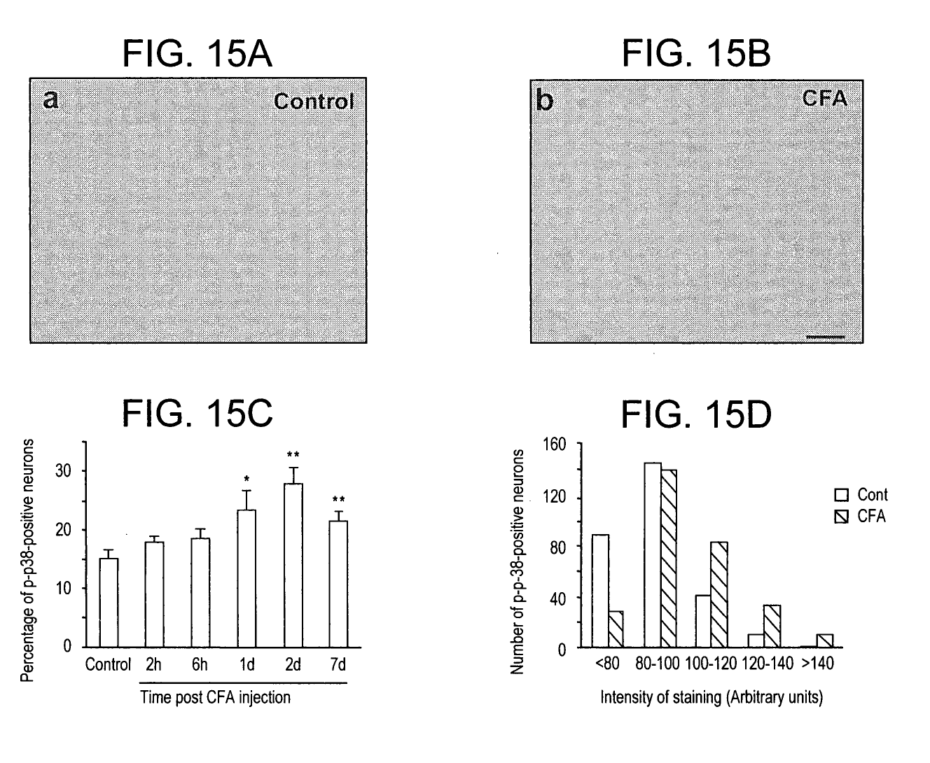

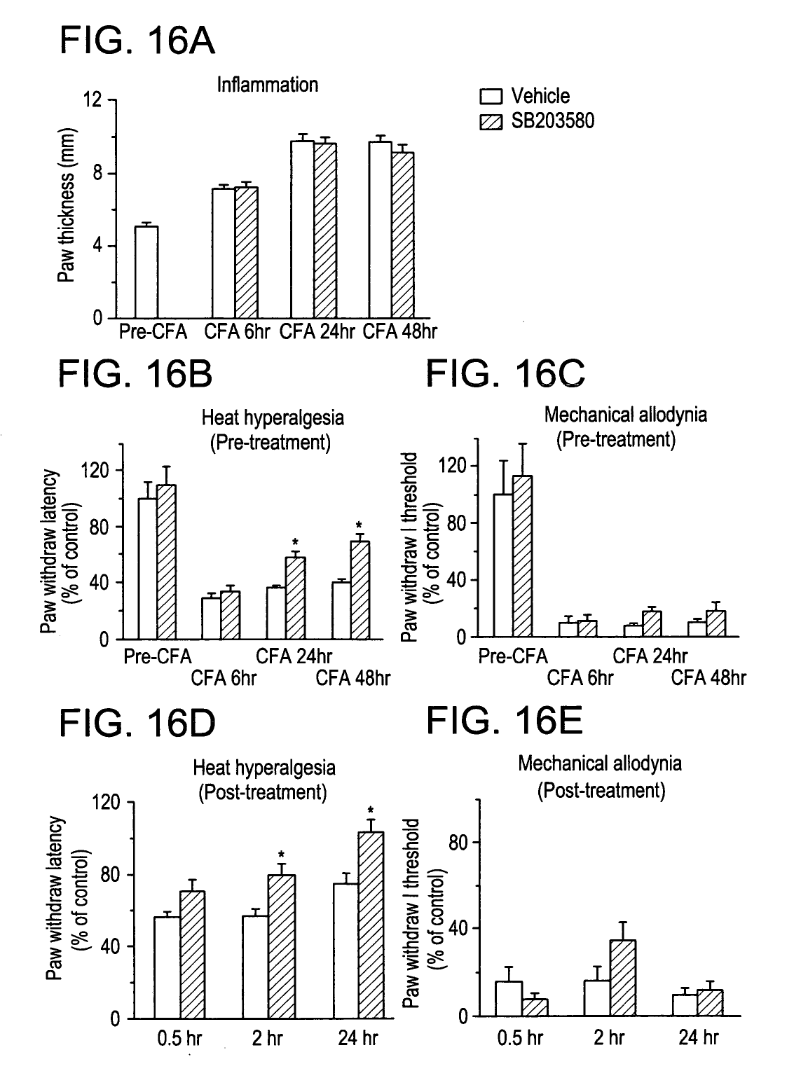

Based on this staining with a phospho-specific p38 antibody, activated p38 (phospho-p38, (p-p38)) is present in around 15% of DRG neurons (all of small size) in native rats. Phospho-p38 is expressed in the nucleus and cytoplasm of neurons and in glial cells. Inflammation induced a substantial increase in the level of phospho-p38 (p-p38) (FIGS. 15A and 15B). This increase was significant one day after CFA injection, reached a peak after two days, and was maintained at a high level for seven days (FIG. 15C). The increase in p-p38 levels manifested not only as the increase in the number of p-p38-immunoreactive neurons, but also as an increase in their intensity (FIGS. 15C and 15D). Around 30% of DRG neurons expressed p-p38 after inflammation. Most of these neurons were of small size, suggesting that they were nociceptors (FIG. 15E). Inflammation did not increase the level of the non-phosphorylated form of p38 in DRG neurons, indicating that the elevation in p-p38 was caused by increased phosphorylation of this MAP kinase rather than by increased expression of the kinase. In both control and inflamed conditions, p-p38 was predominantly expressed in C-fibers. In particular, p-p38 was largely colocalized with the capsaicin receptor VR1 (FIGS. 16C-16E), which is primarily expressed in C-fiber nociceptors. p-p38 did not colocalize with neurofilament-200 (FIGS. 16A and 16B), a marker of A fibers. C-fiber nociceptors can be divided into two groups: NGF-responsive/TrkA expressing neurons and GDNF-responsive/c-ret expressing ones. p-p38 is heavily colocalized with both P2X3 and TrkA in DRG neurons two days after inflammation. Thus, p38 was activated in both types of C-fiber nociceptors after inflammation.

p38 Activation in the Dorsal Horn

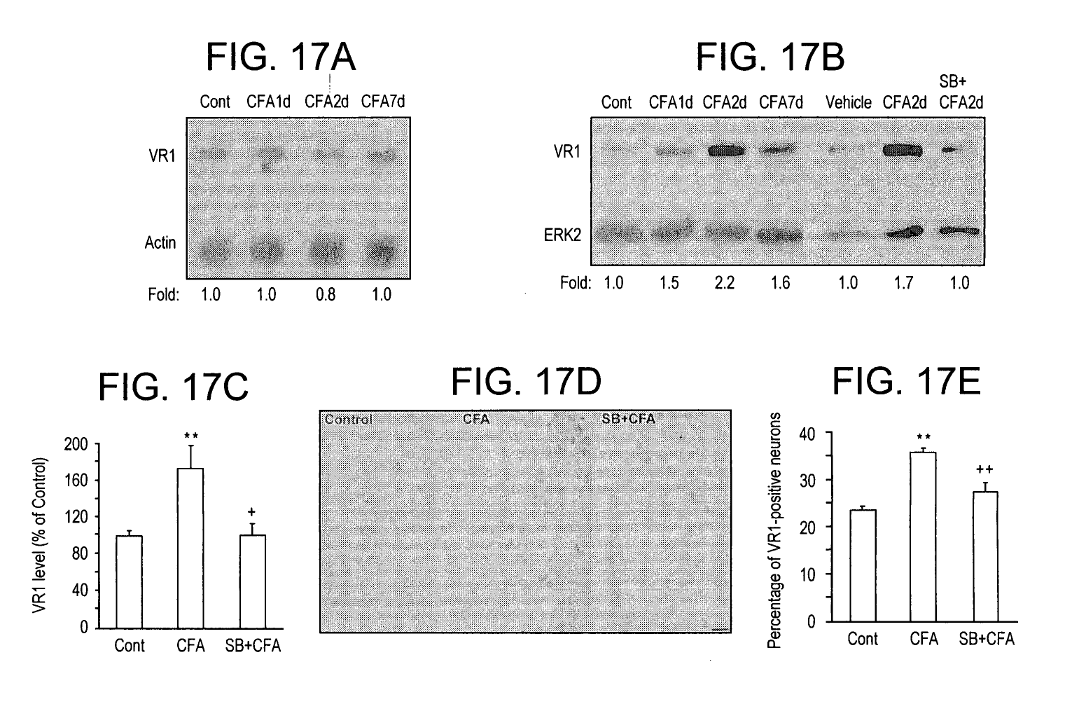

Inflammation leads to a sustained activation of ERK MAP kinase in neurons of the superficial dorsal horn. In contrast, p38 phosphorylation was not increased by inflammation in the dorsal horn (from six hours to seven days). Based on immunohistochemistry analysis performed as described above, p-p38 did not colocalized with NeuN, a neuronal marker (FIGS. 17B and 17C). Thus, p-p38 was expressed in only non-neuronal cells in the dorsal horn in both control and inflamed animals.

p38 Activation and Inflammatory Pain

CFA-induced inflammatory pain is characterized by hyperalgesia (i.e., an increased sensitivity to a noxious stimulus) and allodynia (i.e., generation of pain by a normally innocuous stimulus). To examine the involvement of p38 activation in the DRG in inflammatory pain, 10 μl of a specific p38 inhibitor, SB 203580 (1 μg, Calbiochem), was administered into the intrathecal space (close to L4 DRG level) via an intrathecal PP10 catheter. Although p38 inhibitors have an anti-inflammatory action when delivered systematically, localized intrathecal drug administration should not affect inflammation in the paw. To obtain a sustained and stable drug infusion, an Alzet osmotic pump (seven day pump, 0.5 μl/hour) was filled with the p38-inhibitor SB 203580 (1 μg/μl) in saline, and the associated catheter of the pump was implanted intrathecally sixteen hours before CFA injection. Saline was used as vehicle control for the osmotic pump. Infusion of SB 203085 (0.5 μg/μl/hr) by this approach did not reduce CFA-induced swelling, based on paw thickness (FIG. 16A).

To test whether the p38 inhibitor affects basal pain sensitivity, heat and mechanical sensitivity were measured, as previously described (Ji et al., 2001, supra). Briefly, animals were habituated and basal pain sensitivity was tested before drug administration or surgery. Mechanical withdrawal threshold on the plantar surface of the hindpaw was measured with a set of von Frey hairs. The threshold was taken as the lowest force that evoked a brisk withdraw response. Thermal paw withdrawal latency was measured using the Hargreaves radiant heat apparatus and averaged over three trials. Neither heat nor mechanical sensitivity in non-inflamed rats was affected by SB 203580 (FIG. 16B and 16C). SB 203580 administration did not change the early phase of inflammatory pain (i.e., at a six hour time point) (FIG. 16A). This inhibitor reduced the late phase of inflammation-induced heat hyperalgesia, but did not affect mechanical allodynia (FIGS. 16B and 16C). To investigate whether the p38 inhibitor affects established inflammatory pain, SB203580 (1 μg) was intrathecally injected 48 hours after CFA injection. Pain behavior was measured 0.5, 3, and 24 hours after the inhibitor was injected. SB203580 did not affect inflammatory pain at 0.5 hours, but started to decrease heat hyperalgesia at three hours and completely reversed heat hyperalgesia 24 hours post-injection (FIGS. 16D and 16E). At all time points tested, mechanical allodynia was not altered by the inhibitor (FIGS. 16D and 16E).

p38 Activation and VR1 Expression

An RNase protection assay was used to determine whether expression of VR1 mRNA in DRG neurons was induced by CFA-mediated inflammation. For this assay, L4 and L5 DRGs were rapidly removed. VR1 cDNA was generated by RT-PCR from rat DRG total RNA and cloned into pCRII (Invitrogen). The plasmid was linearized with EcoRV, and an antisense probe was synthesized using Sp6 RNA polymerase and labeled with ³²P-UTP (NEN, 800 Ci/mmol). RNase protection assays were performed using the RPA III (Ambion) protocol, as previously reported (Samad et al., Nature 410: 471-475, 2001). Briefly, 5 μg of RNA samples were hybridized with a labeled probe overnight at 42° C., and then digested with an RNase A/RNase T1 mix in RNase digestion buffer for 30 minutes at 37° C. Finally, samples were separated on a denaturing acrylamide gel and exposed to X-film. A β-actin probe was used for each sample as a loading control. The density of specific bands was measured and normalized with internal control bands from the loading control. The data were represented as mean+SEM. Differences between groups were compared using the student t-test or ANOVA, followed by Fisher's PLSD. For non-parametric data, the Mann-Whittney U test was applied. The criterion for statistical significance was P<0.05. This analysis showed that inflammation does not induce expression of VR1 mRNA in the DRG over the whole time course examined (FIG. 17A).

To determine whether expression of VR1 protein in DRG neurons was induced by peripheral inflammation, the following Western blot analysis was used. The L4 and L5 DRGs and dorsal horns (lumbar enlargement) were lysed in a buffer containing a cocktail of proteinase and phosphotase inhibitors (Sigma). Then, protein samples were separated using a SDS-PAGE gradient gel (4-15%, Bio-Rad) and transferred to PVDF filters. The blots were blocked with 5% milk for one hour and incubated with phosphop38 antibody (1:1000) or VR1 antibody (1:3000) overnight at 4° C. The blots were then incubated in HRP-conjugated secondary antibody (1:3000) for one hour at room temperature, developed in ECL solution (NEN) for one minute, and exposed onto X-film (superfilm) (Amersham) for 2-30 minutes. The blots were then incubated in stripping buffer (100 μM 2-mercaptoethanol, 2% SDS, and 62.5 mM Tris pH 6.7) at 50° C. for 30 minutes and reprobed with anti-p38 and ERK2 antibodies (1:3000, New England Biolab) as a loading control. The density of specific bands was measured and normalized with internal control bands from the loading control. The data were represented as mean+SEM. Differences between groups were compared using the student t-test or ANOVA, followed by Fisher's PLSD. The Mann-Whittney U test was applied to non-parametric data. The criterion for statistical significance was P<0.05. Based on as this Western blot analysis, CFA-induced inflammation induced a sustained increase in VR1 protein levels (FIG. 17B).

To confirm the Western blot results, VR1 immunohistochemistry was performed. Inflammation produced an increase in VR1 immunoreactivity at two days (FIG. 17D). To test whether p38 activation contributes to VR1 upregulation after inflammation, the p38 inhibitor SB 203580 (1 μg) was intrathecally administered (twice a day for two days, with the first injection given 30 minutes prior to CFA injection). This bolus injection decreased the inflammatory heat hyperalgesia and blocked the CFA-induced upregulation of VR1 at two days (FIGS. 17B and 17C). Continuous infusion of SB 203580 via an osmotic pump also decreased the induction of VR1 after inflammation (FIGS. 17D and 17E).

NGF, p38 Activation, and VR1 Expression

After CFA-induced inflammation, NGF is produced in the inflamed paw tissue and is retrogradely transported to the cell body of DRG neurons. NGF is critical for gene expression in DRG neurons and plays a major role in inflammatory pain (Lindsay et al., Nature 337: 362-364, 1989; and Woolf et al., Neuroscience 62:327-331, 1994). NGF has been shown to induce p38 activation in PC 12 cells (Morooka and Nishida, J Biol Chem 273:24385-24288, 1998). The data described above indicates that p38 is activated in TrkA-expressing (NGF responsive neurons) and that p38 leads to VR1 upregulation after inflammation. To determine whether NGF is required for the inflammation-induced activation of p38 and upregulation of VR1, NGF antiserum (i.p., 5 μl/g body weight, 84 mg/ml) was injected to neutralize endogenous NGF. This anti-NGF treatment (once a day for two days, with the first injection one hour prior to CFA injection) substantially reduced inflammatory heat hyperalgesia and reduced mechanical allodynia to a lesser extent. The treatment also decreased the activation of p38 due to administration of CFA and decreased VR1 upregulation. Intrathecal injection of NGF (2 μg in 10 μl, twice a day for three days, Boeringer) increased both p-p38 levels and VR1 levels in DRG neurons. NGF also induced p38 activation in adult primary DRG neurons grown in culture. To test whether NGF acts via p38 to upregulate VR1, the p38 inhibitor SB203580 (1 μg) was co-administrated with NGF (twice a day for three days). SB203580 significantly suppressed the NGF-induced VR1 increase.

### EXAMPLE 6

### Characterization of the Role of ERK MAP Kinase in Pain Hypersensitivity

To further investigate the role of ERK activation in pain hypersensitivity, the activation of ERK by peripheral inflammation was studied. Injection of CFA into a hindpaw produced a persistent inflammation and a sustained ERK activation in neurons in the superficial layers (laminae I-IIo) of the dorsal horn. CFA also induced an upregulation of known pain mediators, prodynorphin and neurokinin-1 (NK-1), in dorsal horn neurons, which was suppressed by intrathecal delivery of the MEK (MAP kinase kinase) inhibitor U0126. CFA-induced pERK largely colocalized with prodynorphin and NK-1 in superficial dorsal horn neurons. While intrathecal injection of U0126 did not affect basal pain sensitivity, it did attenuate both the establishment and maintenance of persistent inflammatory heat and mechanical hypersensitivity. Activation of the ERK pathway in a subset of nociceptive spinal neurons contributes, therefore, to persistent pain hypersensitivity, possibly via transcriptional regulation of genes such as prodynorphin and NK-1. These results further support the role of ERK in pain hypersensitivity and the usefulness of ERK inhibitors in the treatment of acute or chronic pain.

The experiments are described in more detail below.

ERK Activation by Peripheral Inflammation

In order to investigate whether ERK is activated by peripheral inflammation, inflammation was induced in a rat hindpaw by injecting CFA, and phosphorylation of ERK was measured. In particular, adult male Sprague-Dawley rats (230-300 g) were used according to Massachusetts General Hospital Animal Care institutional guidelines. Animals were anesthetized with pentobarbital (50 mg/kg, i.p.). CFA (100 μl) was injected into the plantar surface of a hindpaw. Within an hour of the CFA injection, an area of localized swelling, erythema, and hypersensitivity to mechanical and thermal stimuli, which persisted for the duration of the experiment (48 hours), was produced.

For the immunohistochemistry analysis of the induction in ERK phosphorylation due to the CFA injection, rats were deeply anesthetized with pentobarbital (120 mg/kg, i.p.) and perfused through the ascending aorta with saline followed by 4% paraformaldehyde with 1.5% picric acid. L4-L5 spinal cord segments were dissected and post-fixed for two to four hours. Transverse spinal cord sections (free floating, 30 μm) were cut and processed for immunohistochemistry using the ABC method as described previously (Ji et al., Neuroscience 68:563-576, 1995 and Ji et al., 1999, supra). Briefly, sections were blocked with 2% goat serum in 0.3% Triton for one hour at room temperature, and incubated overnight at 4° C. with primary antibody. The sections were then incubated for two hours with biotinylated secondary antibody (1:200) and one hour with the ABC complex (1:50, Vector Laboratories) at room temperature. Finally, the reaction product was visualized with 0.05% DAB/0.002% hydrogen peroxide in 0.1 M acetate buffer (pH 6.0) containing 2% ammonium nickel sulfate for 2-5 minutes. Some sections were processed with immunofluorescence by incubating overnight with primary antibody and one hour at room temperature with FITC-conjugated secondary antibody (1:300, Jackson immunolab). The following antibodies were used in this analysis: anti-pERK (also called anti-pMAPK; anti-rabbit, 1:500, New England BioLabs) and anti-pERK (monoclonal, 1:300, New England Biolabs). For quantitation of the results from this experiment, eight non-adjacent sections from the L4-L5 lumbar spinal cord were randomly selected, and the numbers of immunoreactive neuronal profiles in the superficial laminae and/or deep laminae of the dorsal horn in each section were counted (under a 20× object field) by an observer blind to the treatment. The values from the eight sections were averaged for each animal. The data are represented as mean+SEM.

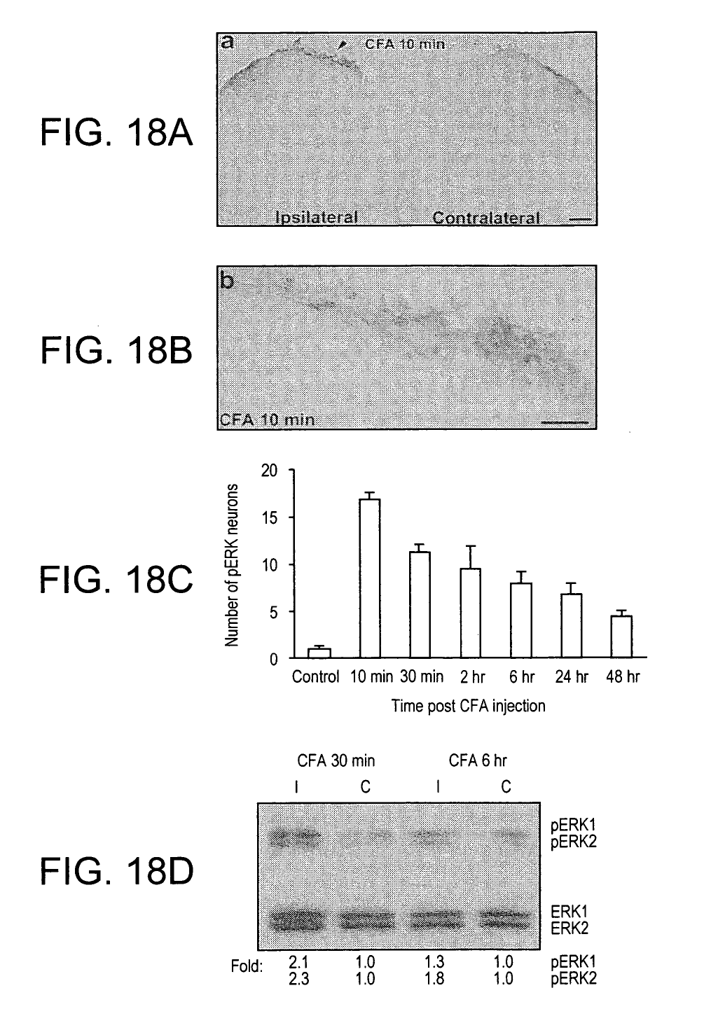

Based on this immunohistochemistry analysis, the inflammation induced by the CFA injection resulted in the induction of phospho-ERK (PERK) in neurons in the medial superficial dorsal horn on the ipsilateral side of the lumbar enlargement (FIGS. 18A and 18B). No induction was found in the contralateral spinal cord (FIG. 18A). Intraplantar saline injections (100 μl) only induced a very weak pERK induction. The CFA-induced pERK induction was found only in neurons; all pERK cells expressed NeuN, a marker for neuronal cells. The pERK labeled neurons were predominantly localized in laminae I-IIo, and pERK was present in the nucleus, cytoplasm, and dendrites, as previously reported (Ji et al., 1999, supra). The number of pERK neurons peaked at 10 minutes but remained elevated with a slow decline, for 48 hours (FIG. 18C). This temporal pattern differs substantially from the transient (<1 hour) ERK activation evoked by intraplantar capsaicin (Ji et al., 1999, supra).

ERK activation by CFA was confirmed by Western blot analysis. For this analysis, animals were sacrificed, and dorsal horns of the L4-L5 spinal segments were rapidly removed and homogenized with a hand-held pellet pestle in lysis buffer containing a cocktail of phosphatase inhibitors (100×) and proteinase inhibitors (25×, Sigma). Protein samples were separated on a SDS-PAGE gel (4-15% gradient gel, Bio-Rad) and transferred to PVDF filters (Millipore). The filters were blocked with 3% milk and incubated overnight at 4° C. with polyclonal anti-pERK (1:1000, New England Biolabs). The blots were incubated for one hour at room temperature with HRP-conjugated secondary antibody (Amersham, 1:3000) and visualized in ECL solution (NEN) for one minute and exposed onto hyperfilms (Amersham) for 1-30 minutes. The blots were then incubated in stripping buffer (67.5 mM Tris, pH 6.8, 2% SDS, 0.7% β-mercaptoethanol) for 30 minutes at 50° C. and reprobed with polyclonal anti-ERK as a loading control. This Western blot analysis was repeated at least twice, and in all cases the same results were obtained. The density of specific bands was measured with a computer-assisted imaging analysis system (IP lab software) and normalized against a loading control. Differences between groups were compared using student the t-test or ANOVA, followed by Fisher's PLSD. For non-parametric data, the Mann-Whittney U test was applied. The criterion for statistical significance was P<0.05.

Based on this Western analysis, the phosphorylation level of both ERK1 (44 kD) and ERK2 (42 kD) increased in the ipsilateral dorsal horn compared to the contralateral side (FIG. 18D). As ERK is only activated in a small subset of dorsal horn cells, Western analysis is less sensitive than immunohistochemistry in detecting ERK activation in the superficial dorsal horn.

Because pERK reached a peak level very rapidly after the CFA injection (10 minutes), we determined whether CFA also produced hyperalgesia or pain-related behavior. For these studies, animals were habituated to the testing environment daily for two days before baseline testing. Except for the heat test, all the animals were placed on an elevated wire grid. For mechanical allodynia, the plantar surface of the hindpaw was stimulated with a series of von Frey hairs. The threshold was taken as the lowest force that evoked a brisk withdrawal response. For heat hyperalgesia, the plantar surface of a hindpaw was exposed to a beam of radiant heat through a transparent Perspex surface (Hargreaves et al., Pain 32:77-88, 1988). The withdrawal latency was recorded, with a maximum 15 seconds as cutoff. The withdrawal latency was averaged over three trials.

In this behavior studies, CFA (100 μl) injected into the plantar surface of hindpaw in awake rats produced both immediate erythema and a rapid heat hyperalgesia. The paw withdrawal latency decreased by 60% (from 10.8±0.4 to 4.3±0.7, P<0.01, t-test, n=3) and 50% (from 9.7±1.2 to 4.9±1.3 P<0.05) at 10 and 30 minutes, respectively. Saline injected rats did not show any heat hypersensitivity.

Prodynorphin Induction by Inflammation

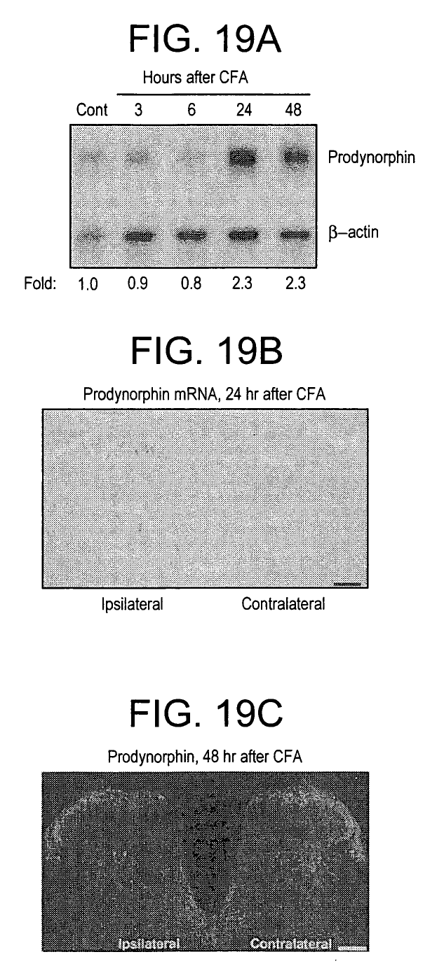

To investigate changes in the expression of prodynorphin in response to inflammation, an RNase protection assay, in situ hybridization, and immunohistochemistry were used. For the RNase protection assay, dynorphin cDNA was generated by room temperature-PCR from rat DRG total RNA, using primers 5′-TGGAAAAGCCCAGCTCCTAGACCCT-3′ (SEQ ID NO: 3) and 5′-TTCCTCGTGGGCTTGAAGTGTGAAA-3′ (SEQ ID NO: 4) and cloned into pCRII (Invitrogen). The plasmid was linearized with EcoRV, and an antisense probe was synthesized using Sp6 RNA polymerase and labeled with 32P-UTP (NEN, 800 Ci/mmol). RNase protection assays were performed using the RNase protection assay III (Ambion) protocol, as previously reported (Samad et al., Nature 22:471-475, 2001). Briefly, 10 μg of RNA samples were hybridized with labeled probe over night at 42° C., then digested with RNase A/RNase T1 mix in RNase digestion buffer for 30 minutes at 37° C. Finally samples were separated on a denaturing acrylamide gel and exposed to X-films. A β-actin probe was used for each sample as a loading control. For RNase protection assay, each experiment was repeated at least twice, and in all cases the same results were obtained. The density of specific bands was measured with a computer-assisted imaging analysis system (IP lab software) and normalized against a loading control. Differences between groups were compared using student the t-test or ANOVA, followed by Fisher's PLSD. For non-parametric data, the Mann-Whittney U test was applied. The criterion for statistical significance was P<0.05. Based on this RNase protection assay, peripheral inflammation resulted in a substantial upregulation of prodynorphin mRNA in the ipsilateral spinal dorsal horn 24 and 48 hours after CFA injection (FIG. 19A).

For the in situ hybridization analysis, animals were rapidly sacrificed in a CO₂ chamber, and L4-L5 spinal cord segments were removed and cut on a cryostat at a thickness of 20 μm. A vector (pSP65) with a 1.7 kb prodynorphin insert was kindly provided by Dr. Linda Kobierski (Harvard Medical School). An antinsense RNA probe, and the corresponding sense control probe, were labeled by in vitro transcription using linearized DNA templates for prodynorphin and dig labeling mixture for two hours at 37° C. Hybridization was processed as previously described (Ji et al., Proc Natl Acad Sci USA 95:15635-15640, 1998). Tissue Sections were air dried for two hours, fixed in 4% paraformaldehyde for 15 minutes, and acetylated in acetic anhydride (0.25%) for 10 minutes. Sections were pre-hybridized for two hours at room temperature, then incubated in hybridization buffer overnight at 60° C. After hybridization, sections were washed in decreasing concentrations of SSC (2×, 1×, and 0.2×) for two hours total. Sections were then blocked with 2% goat serum for one hour and incubated overnight at 4° C. with alkaline phosphatase-conjugated anti-DIG antibody (Boehringer Mannheim, 1:5000). Finally the sections were visualized in 75 g/ml NBT, 50 g/ml BCIP, and 0.24 mg/ml levamisole for two to twenty-four hours. Eight non-adjacent sections from the L4-L5 lumbar spinal cord were randomly selected, and the numbers of mRNA-positive neuronal profiles in the superficial laminae and/or deep laminae of the dorsal horn in each section were counted (under a 20× object field) by an observer blind to the treatment. The values from the eight sections were averaged for each animal. The data are represented as mean+SEM. Based on this in situ hybridization a nalysis, many strongly labeled prodynorphin mRNA labeled neurons were found both in the superficial and deep layers of the ipsilateral dorsal horn 24 hours post CFA injection; whereas on the contralateral side, only a few weakly labeled neurons were detected (FIG. 19B).

Imnuunohistochemistry was performed as described above using the anti-prodynorphin antibody (anti-guinea pig, 1:3000, kindly provided by Dr. R. Elde, University of Minnesota). An increase in the number of prodynorphin peptide immunoreactive neurons was also found in the superficial and deep dorsal horn 48 hours after the CFA-induced inflammation (FIG. 19C).

ERK Activation and Prodynorphin Expression

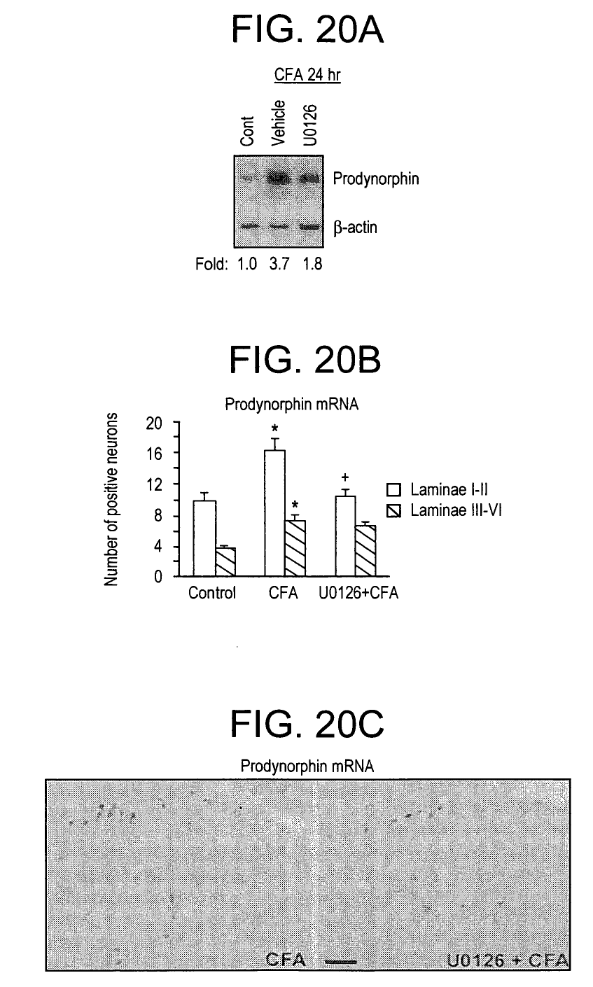

The possible regulation of prodynorphin mRNA expression in the dorsal horn by ERK activation was investigated. A specific and potent MEK inhibitor U0126 (Favata et al., J Biol Chem 273 :18623-18632, 1998) was intrathecally injected twice (1 μg), 30 minutes before and 6 hours after intraplantar CFA injection. For administration, a PE10 catheter was implanted into the intrathecal space of the spinal cord at the lumbar enlargement and 10 μl of MEK inhibitor U0126 (1 μg, Calbiochem, dissolved in 10% DMSO) was administered. Ten percent DMSO was injected as a vehicle control. This inhibitor reduced the CFA-induced prodynorphin mRNA increase in the ipsilateral dorsal horn (FIG. 20A). The CFA-evoked increase in the number of prodynorphin mRNA-positive neurons in the superficial dorsal horn was also decreased by U0126 (2×1 μg) without affecting the number of labeled neurons in the deep laminae (FIGS. 20B and 20C).

ERK Activation and NK-1 Expression

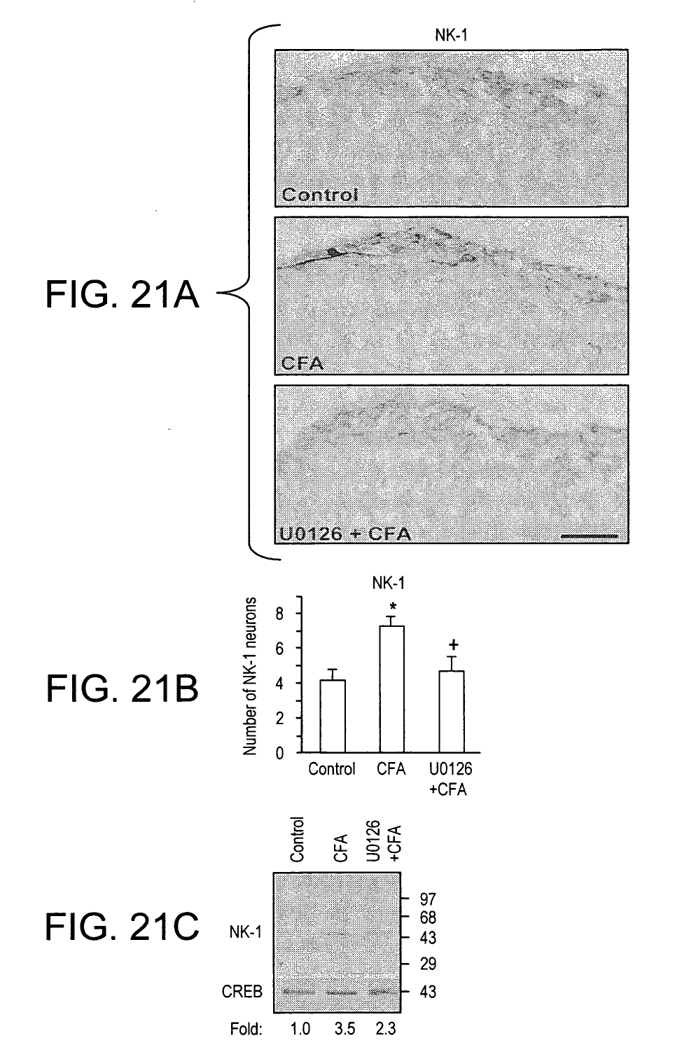

In agreement with earlier studies, increased NK-1 immunoreactivity in the superficial dorsal horn after CFA-induced inflammation was observed using the anti-NK1 antibody (anti-rabbit, 1:3000, Oncogene) in the immunohistochemistry assay described above (Abbadie et al., Neuroscience 70:201-209, 1996, and Abbadie et al., J Neurosci 17: 8049-8060 1997). However, in contrast to the previous studies (Abbadie et al., 1997 supra; and Honore et al., J Neurosce 19:7670-7678, 1999), we found more NK-1 expressing cells after inflammation (FIG. 21A). This discrepancy is almost certainly due to the different detection thresholds for NK-1-positive neurons; our quantification, based on standard immunofluorescence microscopy, did not include weakly stained cells in control animals, cell that would be detected with confocal microscopy (Abbadie et al., 1997 supra; Honore et al., 1999, supra). The increase in NK-1 immunoreactive neurons that was detected in lamina I (FIG. 21A), reflects the increase in staining intensity seen by Honore et al. (1999, supra) in this lamina. To explore whether ERK activation is involved in NK-1 upregulation, the MEK inhibitor U0126 was delivered intrathecally before the induction of inflammation via an osmotic pump (0.5 μg/ul/hr for 2 days). In particular, an Alzet osmotic pump (three days pump, 1 μl/hr) was filled with the MEK-inhibitor U0126 (0.5 μg/μl) in 50% DMSO, and the catheter of the pump implanted intrathecally at least three hours before CFA injection. DMSO (50%) was used as vehicle control. MEK inhibition suppressed the CFA-induced elevation of NK-1 immunoreactive neurons in the superficial dorsal horn (FIGS. 21A and 21B). Western blot analysis using the anti-NK1 (1:5000, Oncogene) primary antibody with the anti-CREB antibody (1:3000, New England Biolab) as a loading control also confirmed this result (FIG. 21C).

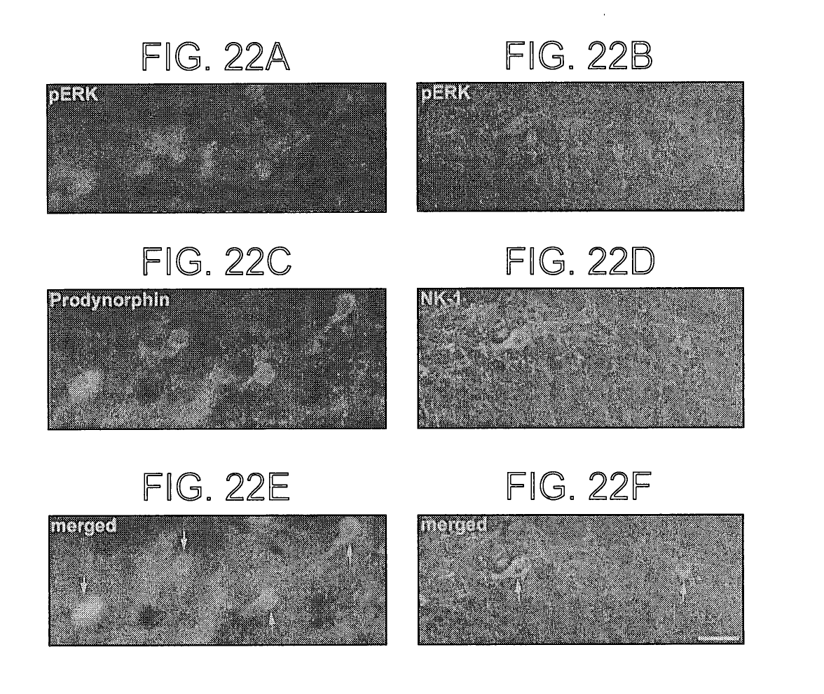

To test whether the pERK-positive neurons and prodynorphin-/NK-1-expressing neurons belong to the same subset of dorsal horn cells, double immunofluorescence for pERK/prodynorphin and for pERK/NK-1 was performed. This immunofluorescence analysis was conducted by incubating a mixture of primary antibodies (monoclonal anti-pERK/polyclonal anti-NK1 or rabbit anti-pERK/guinea pig anti-prodynorphin) followed by a mixture of corresponding secondary antibodies conjugated with either Cy3 or FITC. Almost all prodynorphin- and NK-1 positive neurons in the superficial dorsal horn also expressed pERK 24 hours after CFA injection (FIGS. 22A-H).

ERK Activation and Persistent Inflammatory Pain

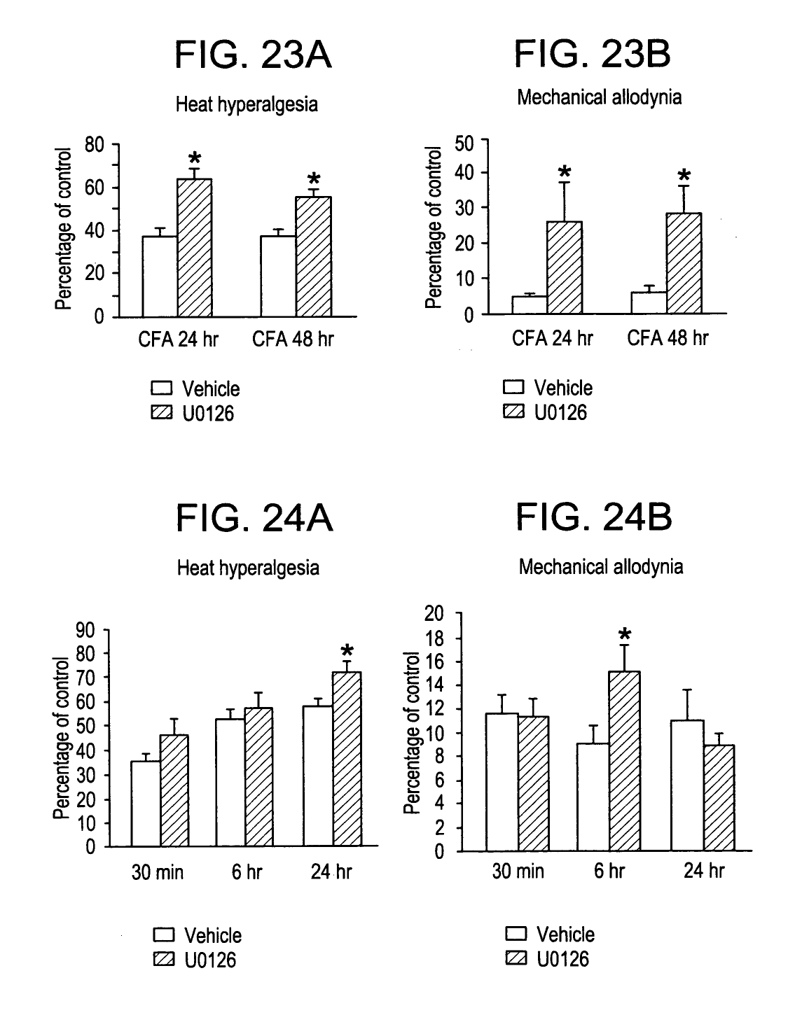

To examine the functional consequences of ERK activation and its downstream effects on prodynorphin and NK-1 upregulation, we determined whether inhibition of ERK activation modified inflammatory pain hypersensitivity. Intrathecal administration of U0126 (1 μg) into non-inflamed animals, like another MEK inhibitor PD 98059 (Ji et al., 1999, supra), produced no significant change in basal pain sensitivity measured in terms of mechanical withdrawal threshold (108% of the vehicle control) and heat withdrawal latency (113% of vehicle control), when tested 30 minutes after the administration. However, intrathecal administration of U0126 via an osmotic pump (0.5 μg/μl/hr), started before the CFA injection and maintained for 48 hours, significantly reduced the inflammation-induced heat and mechanical hypersensitivity measured at 24 and 48 hours (FIGS. 23A and 23B).

Acute pain hypersensitivity (10-60 minutes after an intraplantar formalin injection) is reduced by inhibition of ERK activation, presumably by preventing post-translational changes (Ji et al, 1999, supra). ERK activation by CFA may contribute to inflammatory pain hypersensitivity either by maintaining ongoing post-translational changes or by inducing transcription of genes such as NK1 and prodynorphin. In the former case, blocking ERK activation in established inflammation would be expected to reduce the pain hypersensitivity within tens of minutes due to dephosphorylation of ERK substrates. If the contribution of ERK activation were through transcription, inhibiting ERK activation would be expected to have no immediate effect, but have a delayed effect. To distinguish between these two possible mechanisms, U0126 was intrathecally injected (1 μg) into rats with established inflammation (24 hours after CFA injection) and tested pain hypersensitivity 30 minutes, 6 hours, and 24 hours after the injection of U0126. Neither heat hyperalgesia nor mechanical allodynia was significantly affected by such post-treatment when tested at 30 minutes (FIGS. 24A and 24B). However, the post-treatment decreased heat hyperalgesia at 24 hours and mechanical allodynia at 6 hours (FIGS. 24A and 24B), indicating a delayed contribution of ERK activation to the maintenance of persistent inflammatory pain.

ERK Activation in Nociceptive Dorsal Horn Neurons

Peripheral inflammation induced, after a short latency, a persistent activation of ERK in laminae I-IIo neurons of the ipsilateral superficial dorsal horn. Inhibition of this activation, using a MEK inhibitor, blocked elevation of prodynorphin and NK-1 expression in this particular subset of dorsal horn neurons and reduced inflammatory pain hypersensitivity. pERK was induced by CFA in the same subset of dorsal horn neurons that express prodynorphin and NK-1. Many NK-1 and dynorphin expressing neurons in lamina I are projection neurons (Marshall et al., Neuroscience 72, 255-263, 1996; and Nahin et al., Neurosci Lett 96:247-252, 1989). Projection neurons in lamina I exhibit an enlargement of their receptive fields after CFA-induced inflammation (Dubner et al., Trends Neurosci 15:96-103, 1992 and Ruda, Proc Natl Acad Sci USA 85:622-626, 1992) and a targeted loss of NK-1 expressing neurons in lamina I has been shown to abolish inflammatory pain (Nichols, et al., Science 286:1558-1561, 1999), indicating a critical role for these superficial neurons in the reaction of the CNS to inflammation. A particular subset of C-nociceptor fibers, those that are NGF-responsive, and TrkA and neuropeptide expressing, terminate in lamina I and IIo, in an area overlapping the neurons that show ERK activation. Another subset of C-fibers, those that respond to the GDNF family of growth factors and are characterized by selective binding of the IB4 lectin, terminate in lamina IIi (Averill et al., Eur J Neurosci 7:1484-1494, 1995; Moliver et al., 1997). The neurons these fibers contact, many of which contain PKC((Malmberg et al., Science 278:279-283, 1997), do not show ERK activation after capsaicin or CFA injection. ERK's role in regulating pain hypersensitivity is, therefore, restricted to a particular subset of nociceptive dorsal horn neurons, only those located in laminae I-IIo, and this activation is likely to reflect activation only of TrkA-expressing C-fibers.

Transcriptional Regulation in Response to ERK Activation

pERK was found in the nucleus of neurons after CFA stimulation (FIG. 18A), pointing to a possible transcriptional role for the activated kinase. Unlike the transient activation (lasting less than 2 hours) induced after capsaicin injection (Ji et al., 1999, supra), CFA produced persistent ERK activation (FIG. 18C). The sustained ERK activation after CFA injection is associated with persistent upregulation of prodynorphin mRNA (lasting more than 48 hours, FIG. 19A); whereas the transient pERK induced by capsaicin is associated with a shorter lasting upregulation of prodynorphin mRNA (<6 hours). ERK activation is likely to regulate the expression of prodynorphin and NK1, both of which are CRE-containing genes, via CREB phosphorylation. CREB is required for dopamine-induced expression of prodynorphin in striatal neurons (Cole et al., 1995, supra) and is phosphorylated in NK-1 receptor expressing neurons after noxious stimulation (Anderson et al., Neurosci Lett 283:29-32, 2000 and Seybold, 2000). Furthermore, a CRE site has been shown to mediate a long-term sensitization of nociceptive neurons in Aplysia (Lewin et al., Nat. Neurosci. 2, 18-23, 1999).

ERK Activation and Inflammatory Pain Hypersensitivity

U0126 is a potent and selective MEK inhibitor (Favata et al, 1998, supra) that can inhibit ERK activation even in the presence of strong activators such as phorbol esters, but does not affect other signal transduction pathways. At the dose used in the present studies, obvious signs of toxicity due to this inhibitor were not observed: animals behaved normally and locomotion was unaffected. Although basal pain sensitivity was not modified by the inhibitor, persistent inflammatory pain was reduced. This effect on persistent pain may result from either post-translational change mediated by the ERK signal transduction pathway or by a reduction of transcription of target genes such as prodynorphin and NK1. The regulation of prodynorphin and NK1, which have been previously implicated in pain mechanisms, by ERK activation is compatible with the hypothesis that ERK activation following inflammation contributes to pain hypersensitivity by regulating gene transcription. The temporal profile of the effect of blocking ERK activation further support this hypothesis. In contrast, the acute pain hypersensitivity established within minutes of intraplantar formalin can be reduced by preventing ERK activation (Ji et al, 1999, supra); this effect is too quick (<1 hour) to be mediated by an inhibition of transcription and is likely therefore to represent a post-translational change downstream of activated ERK. The substrate for such a post-translational change may be an ion channel or receptor such as the NMDA or AMPA receptor (Woolf et al., 2000, supra). Such post-translational changes underlie the induction and maintenance of central sensitization, a use-dependent plasticity that outlasts its initiating stimulus by tens of minutes (Woolf et al., Nature 306:686-688, 1983; and Woolf et al., J Neurosci 6:1433-1442, 1986). If inflammatory hypersensitivity were a manifestation only of a central sensitization maintained by ongoing afferent input from the inflamed tissue, then blocking the initiation of central sensitization by inhibiting an ERK-mediated phosphorylation should reduce the hypersensitivity over a periods of tens of minutes as the key proteins are dephosphorylated. The fact that MEK inhibition during established inflammation had no immediate effect, but rather only reduced mechanical and thermal hypersensitivity 6-24 hours later, argues that the role of ERK activation may well be via transcriptional regulation.

Dynorphin and NK-1 Contribute to Inflammatory Pain Hypersensitivity

A temporal correlation between the expression of prodynorphin and NK-1, and the development of inflammatory pain hypersensitivity has been previously demonstrated. Unlike other opioid peptides, intrathecal injection of dynorphin does not produce analgesia. Dynorphin has been found by others to be pronociceptive in some pathological pain states. For example, dynorphin A antiserum reduces the pain hypersensitivity after nerve injury, and neuropathic pain does not persist in prodynorphin knockout mice. The pronociceptive action of dynorphin appears to be the result of its non-opioid actions, including an activation of NMDA receptors sufficient to induce excitotoxicity.

Inflammation induces NK-1 receptor upregulation in dorsal horn neurons and upregulation of its ligand, the neuropeptide substance P in primary afferent neurons. NK-1 antagonists have been previously shown to reduce inflammatory pain (both hyperalgesia and mechanical allodynia) in several different animal models including NK-1 knock out mice. The increased amount and internalization of the NK-1 receptor on the dendrites of dorsal horn neurons in response to noxious and innocuous stimuli after inflammation indicates that this receptor is activated by substance P in response to peripheral stimuli.

### EXAMPLE 7

### No Induction of PLA, in the Central Nervous System

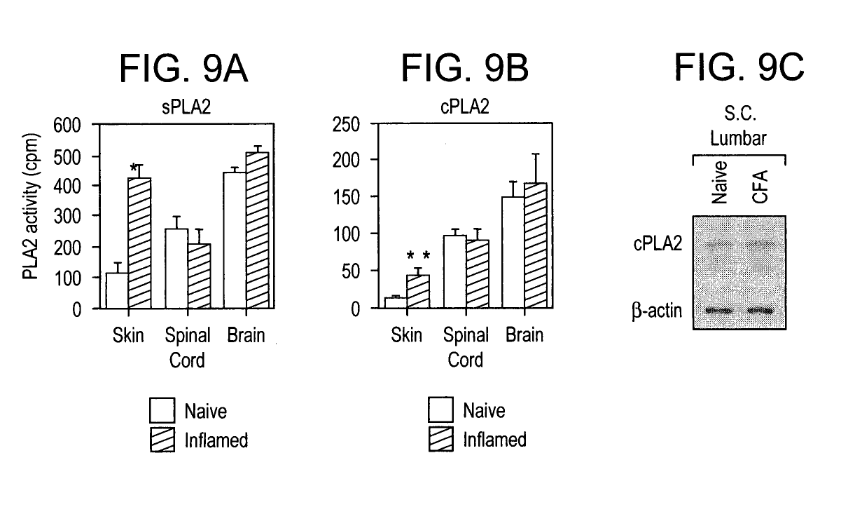

Because phospholipase₂ (PLA₂) regulates the availability of free arachidonic acid which is required for the synthesis of PGE₂, we also determined whether small molecular weight, secreted PLA₂s (sPLA₂s) or large molecular weight, intracellular cytosolic PLA₂ (cPLA₂) are induced in the central nervous system following peripheral inflammation. PLA₂ activities in total protein extracts from the paw, spinal cord, and the brain of naive and inflamed rats (12 hours) were measured using assay systems favoring either the activity of sPLA₂ (i.e., the release of arachidonic acid from 2-arachidonyl-phosphatidylcholine; FIG. 9A) or the activity of cPLA₂ (i.e., the release of arachidonic acid from 2-arachidonyl-phosphatidylethanolamine; FIG. 9B) (Bingham et al., J. Biol. Chem. 274:31476-31484, 1999). As expected, there were significant increases in both sPLA₂ and cPLA₂ activities in the inflamed paw (FIGS. 9A and 9B). In contrast, no increase in these PLA₂ activities was detected in either the spinal cord or the brain due to hindpaw inflammation. Western blot and immunohistochemical analysis of cPLA₂ using a polyclonal antibody against the first 129 amino acid residues of cPLA₂ (Sapirstein et al., J. Biol. Chem. 271:21505-21512, 1996) also failed to show protein induction within the spinal cord (FIG. 9C). Therefore, the basal level of central PLA₂ activity is sufficient for the increased production of prostanoids in the central nervous system after peripheral inflammation. Thus, in contrast to the peripheral site of inflammation where the rate of prostanoid production is regulated by both Cox-2 and PLA₂, Cox-2 alone appears to have the pivotal role for central PGE₂ induction.

### EXAMPLE 8

### Assays for Inhibitors of Central IL-1 Activity

Inhibition of central IL-1β activity may be identified by any standard method for measuring pain (for example, those methods described herein) or by any method for measuring changes in IL-1β activity (for example, as described above). In addition, one mechanism by which central IL-1β activity may be inhibited is through inhibition of caspase-1, which converts pro-interleukin-1β to the active form of IL-1β. Many caspase-1 inhibitors contain a Tyr-Val-Ala-Asp, Val-Ala-Asp, Ala-Asp, or Asp peptide recognition sequence attached to a functional group such as an aldehyde, chloromethylketone, fluoromethylketone, fluoroacyloxymethylketone, diazomethylketone, or phenylalkylketone. Caspase-1 inhibitors with an aldehyde group are reversible, while those with chloromethylketone, fluoromethylketone, or fluoroacyloxymethylketone groups are irreversible (Calbiochem Technical Bulletin entitled Caspase Inhibitors and Substrates, San Diego, Calif.). Inhibitors with a long hydrophobic region, such as the inhibitor Ac-Ala-Ala-Val-Ala-Leu-Leu-Pro-Ala-Val-Leu-Leu-Ala-Leu-Leu-Ala-Pro-Tyr-Val-Ala-Asp-aldehyde, have increased cell permeability. Esterification of the carboxylic side chain of the aspartic acid residue using standard techniques to replace the hydroxyl group of the carboxylic acid with an alkoxy group, such as a methoxy, ethoxy, benzoxy, or isopropoxy group, also increases cell permeability of caspase-1 inhibitors. Preferred alkoxy groups have the formula —OR′, wherein R′ is an alkyl or aryl group. In one preferred embodiment, the R′ group is a linear or branched saturated hydrocarbon alkyl group of 1 to 10, 1 to 20, 1 to 50, or 1 to 100 carbon atoms; such as a methyl, ethyl, n-propyl, isopropyl, n-butyl, isobutyl, t-butyl, octyl, decyl, or tetradcyl group; or a cycloalkyl group, such as a cyclopentyl or cyclohexyl group. Preferred aryl groups include monovalent aromatic hydrocarbon radicals consisting of one or more fused rings in which at least one ring is aromatic in nature, which may optionally be substituted with one of the following substituents: hydroxy, cyano, alkyl, alkoxy, thioalkyl, halo, haloalkyl, hydroxyalkyl, nitro, amino, alkylamino, or diakylamino.

To test candidate caspase-1 inhibitors, caspase-1 activity may be assayed in the presence and absence of the candidate compound to determine the rate of cleavage of a caspase-1 substrate that contains either a fluorophore (e.g., AFC, AMC, EDANS, or MCA) or a chromophore (e.g., pNA) as described previously (Thornberry et al., Nature 356:7680774, 1992; Thornberry et al., Biochemistry 33:3934-3940, 1994). Additionally, candidate caspase-1 inhibitors may be tested in any of the inflammatory pain models described herein to determine their ability to reduce, stabilize, prevent, or delay the onset of pain.

Candidate MAP kinase inhibitors can be tested by measuring their inhibition of the phosphorylation of one or more MAP kinases using a phospho-specific antibody, as described herein. Alternatively, the ability of a MAP kinase to phosphorylate a substrate can be measured in the presence and absence of the candidate compound using standard kinase assays.

### Other Embodiments

From the foregoing description, it will be apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A method for treating, reducing, or preventing pain comprising contacting the central nervous system of a mammal with a first compound that decreases IL-1β activity in an amount sufficient to treat, reduce, or prevent said pain, wherein said compound decreases the level of IL-1β mRNA or protein, an activity of IL-1β, the half-life of IL-1β mRNA or protein, or the binding of IL-1β to a receptor or to another molecule.

2. The method of claim 1, wherein said contacting comprises administering said compound to the central nervous system of said mammal.

3. The method of claim 2, wherein said compound is administered intrathecally, intramedullarly, intracerebrally, intracerebroventricularly, intracranially, epidurally, intraspinally, or intraparietally.

4. The method of claim 1, wherein said compound crosses the blood-brain barrier of said mammal.

5. The method of claim 4, wherein said contacting comprises administering said compound intravenously, parenterally, subcutaneously, intramuscularly, ophthalmically, intraventricularly, intraperitoneally, orally, topically, or intranasally to said mammal.

6. The method of claim 1, wherein said mammal is a human.

7. The method of claim 1, wherein said compound is an IL-1 receptor antagonist or a caspase-1 inhibitor.

8. The method of claim 1, wherein said method further comprises administering said first compound or a second compound to the periphery or central nervous system of said mammal, wherein said first or said second compound inhibits the phosphorylation or activity of p38 or ERK.

9. A method for treating, reducing, or preventing pain comprising contacting the central nervous system of a mammal with a compound that decreases the enzymatic activity or phosphorylation level of p38 in an amount sufficient to treat, reduce, or prevent pain.

10. The method of claim 9, wherein said contacting comprises administering said compound to the central nervous system of said mammal.

11. The method of claim 10, wherein said compound is administered intrathecally, intramedullarly, intracerebrally, intracerebroventricularly, intracranially, epidurally, intraspinally, or intraparietally.

12. The method of claim 9, wherein said method further comprises administering a compound that inhibits the phosphorylation or activity of ERK to the periphery or central nervous system of said mammal.

13. The method of claim 9, wherein said mammal is a human.

14. A method for treating, reducing, or preventing pain comprising contacting the periphery of a mammal with a first compound that decreases the enzymatic activity or phosphorylation level of a first MAP kinase in an amount sufficient to treat, reduce, or prevent pain.

15. The method of claim 14, wherein said contacting comprises administration of said compound intravenously, parenterally, subcutaneously, intramuscularly, ophthalmically, intraventricularly, intraperitoneally, orally, topically, or intranasally to said mammal.

16. The method of claim 14, wherein said first MAP kinase is p38 or ERK.

17. The method of claim 14, wherein said method further comprises administering said first compound or a second compound to the central nervous system (CNS) or periphery of said mammal, wherein said second compound decreases the enzymatic activity or phosphorylation level of said first MAP kinase or a second MAP kinase.

18. The method of claim 17, wherein said CNS administration is intrathecal, intramedullar, intracerebral, intracerebroventricular, intracranial, epidural, intraspinal, or intraparietal.

19. The method of claim 17, wherein said second compound is administered peripherally and crosses the blood-brain barrier of said mammal.

20. The method of claim 19, wherein said administration is intravenous, parenteral, subcutaneous, intramuscular, ophthalmic, intraventricular, intraperitoneal, oral, topical, or intranasal.

21. The method of claim 17, wherein said second MAP kinase is p38 or ERK.

22. The method of claim 21, wherein said first MAP kinase is p38 and said second MAP kinase is ERK.

23. The method of claim 21, wherein said first MAP kinase is ERK and said second MAP kinase is p38.

24. The method of claim 14, wherein said mammal is a human.
